SEQUENCE LISTING (20 sequences)

SEQ ID NO: 1 - DNA, 18 bp, Artificial Sequence
  Glut1 Fwd primer
agccctgctacagtgtat

SEQ ID NO: 2 - DNA, 17 bp, Artificial Sequence
  Glut1 Rev primer
aggtctcgggtcacatc

SEQ ID NO: 3 - DNA, 20 bp, Artificial Sequence
  Glut3 Fwd primer
atggggacaacgaaggtgac

SEQ ID NO: 4 - DNA, 21 bp, Artificial Sequence
  Glut3 Rev primer
caggtgcattgatgactccag

SEQ ID NO: 5 - DNA, 20 bp, Artificial Sequence
  HIF1alpha Fwd primer
tctcggcgaagcaaagagtc

SEQ ID NO: 6 - DNA, 23 bp, Artificial Sequence
  HIF1alpha Rev primer
agccatctagggctttcagataa

SEQ ID NO: 7 - DNA, 21 bp, Artificial Sequence
  Hexokinase Fwd primer
atgatcgcctgcttattcacg

SEQ ID NO: 8 - DNA, 22 bp, Artificial Sequence
  Hexokinase Rev primer
cgcctagaaatctccagaaggg

SEQ ID NO: 9 - DNA, 19 bp, Artificial Sequence
  Phosphofructokinase Fwd primer
catcgccgtgttgacctct

SEQ ID NO: 10 - DNA, 21 bp, Artificial Sequence
  Phosphofructokinase Rev primer
cccgtgaagataccaactcgg

SEQ ID NO: 11 - DNA, 20 bp, Artificial Sequence
  GAPDH Fwd primer
cccttaagagggatgctgcc

SEQ ID NO: 12 - DNA, 20 bp, Artificial Sequence
  GAPDH Rev primer
actgtgccgttgaatttgcc

SEQ ID NO: 13 - DNA, 21 bp, Artificial Sequence
  Phosphoglycerate kinase Fwd primer
cccagaagtcgagaatgcctg

SEQ ID NO: 14 - DNA, 19 bp, Artificial Sequence
  Phosphoglycerate kinase Rev primer
ctcggtgtgcagtcccaaa

SEQ ID NO: 15 - DNA, 21 bp, Artificial Sequence
  Enolase Fwd primer
agtacgggaaggacgccacca

SEQ ID NO: 16 - DNA, 20 bp, Artificial Sequence
  Enolase Rev primer
gcggccacatccatgccgat

SEQ ID NO: 17 - DNA, 19 bp, Artificial Sequence
  Pyruvate kinase Fwd primer
gccgcctggacattgactc

SEQ ID NO: 18 - DNA, 22 bp, Artificial Sequence
  Pyruvate kinase Rev primer
ccatgagagaaattcagccgag

SEQ ID NO: 19 - DNA, 23 bp, Artificial Sequence
  Beta-actin Fwd primer
ccttcttgggtatggaatcctgt

SEQ ID NO: 20 - DNA, 23 bp, Artificial Sequence
  Beta-actin Rev primer
ggcatagaggtctttacggatgt

(19) United States
(12) Patent Application Publication
(10) Pub. No.: US 2021/0346326 A1
(43) Pub. Date: Nov. 11, 2021

(54) COMPOSITIONS AND METHODS FOR ALTERING NEUTROPHIL MIGRATION AND METASTASIS

(71) Applicant: The Wistar Institute of Anatomy and Biology, Philadelphia, PA (US)

(72) Inventor: Dmitry I. Gabrilovich, Villanova, PA (US)

(21) Appl. No.: 17/284,993

(22) PCT Filed: Oct. 14, 2019
(86) PCT No.: PCT/US19/56124

§ 371 Date: Apr. 13, 2021

Related U.S. Application Data
(60) Provisional application No. 62/745,903, filed on Oct. 15, 2018.

(51) Int. Cl. A61K 31/185; A61K 31/7076; A61K 31/513; A61K 31/438; A61K 31/7084; A61K 31/436; A61K 45/06; G01N 33/574

(52) U.S. Cl. CPC A61K 31/185; A61K 31/7076; A61K 31/513; G01N 33/57484; A61K 31/7084; A61K 31/436; A61K 45/06; A61K 31/438

(57) ABSTRACT

A method of identifying a population of PMN-MDSC-Like Cells (PM-LCs) distinguishable from myeloid derived suppressor cells (PMN-MDSCs). A method for treating a cancer comprising identifying PM-LCs and/or administering an agent that alters neutrophil migration to reduce or inhibit tumor cell seeding, tumor metastasis, or metastatic niche formation in a subject. Compositions for use in diagnosing and treating cancer that comprise antagonists and/or inhibitors of genes and cellular process to alter PM-LC activity.

### STATEMENT OF GOVERNMENT SUPPORT

This invention was made with government support under grant numbers P01 CA140043 and T32 CA09171 awarded by the National Institutes of Health. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “WST178PCT_ST25.txt”, was created Oct. 11, 2019, and is 5 KB in size.

### BACKGROUND OF THE INVENTION

The role of neutrophils in cancer is controversial, which is the result of seemingly contradictory activity of these cells able to either promote tumor growth or exert antitumor effects^{1, 2, 3, 4, 5}. Identification of polymorphonuclear myeloid-derived suppressor cells (PMN-MDSCs), pathologically activated neutrophils accumulating in cancer that are characterized by immune-suppressive and pro-tumorigenic activity, helped to partially resolve this controversy³. It is also suggested that some neutrophils that accumulated in cancer and chronic inflammation contribute to tumor development and progression without eliciting immunosuppressive activity^{6,7} . These cells were provisionally termed ‘MDSC-like’ cells⁸. However, the characteristics of these cells and their distinction from control neutrophils in tumor-free hosts have yet to be defined.

Metastasis, or dissemination of tumors to sites distant from the primary tumor, is the leading cause of mortality in cancer⁹. There is strong evidence to support the role of neutrophils and PMN-MDSCs in tumor metastasis^{4, 10, 11, 12, 13}. PMN-MDSCs can condition tumor cells at the primary site to facilitate metastasis, possibly through pathways that regulate the production of hepatocyte growth factor and TGF-β to induce tumor epithelial-mesenchymal transition¹⁴, the production of matrix metalloproteinase 9 to facilitate tumor invasion^{15, 16}, direct immunosuppressive activity that promotes metastasis¹³, and by tethering tumor cells to the vascular endothelium to promote lung metastasis¹⁷. The mechanisms regulating formation of the pre-metastatic niche by neutrophils and PMN-MDSC are much less clear. S100A8 and S100A9 proteins are known to drive the recruitment of PMNs and PMN-MDSC to pre-metastatic sites in colon cancer patients, and PMN, via the production of S100 proteins, can create a positive feedback loop leading to the accumulation of more PMN in the pre-metastatic lung ^{18, 19}. However, the mechanism of initial events leading to formation of the feedback loop remained unclear.

A fundamental characteristic of neutrophils is their ability to migrate to sites of inflammation. This process is directed by chemokines, danger-associated molecule pattern molecules, lipid metabolites, and others^{20, 21}. However, it is not clear what would drive initial neutrophil migration to an uninvolved, distant site preceding tumor cells in the absence of measurable inflammation. In addition, although recruitment of PMN-MDSC to the tumor site is well-documented²², their migration to other uninvolved tissues was not clear. Moreover, it was reported that some PMN-MDSCs have dramatically reduced migratory activity²³.

### SUMMARY OF THE INVENTION

In one aspect, a method of preventing or decreasing metastasis in a subject in need thereof is provided. The method includes administering an effective amount of an agent that alters neutrophil migration to reduce or inhibit tumor cell seeding, tumor metastasis, or metastatic niche formation in the subject.

In another aspect, a method for a treating a cancer is provided. The method includes administering an effective amount of an agent that alters neutrophil migration to reduce or inhibit tumor cell seeding, tumor metastasis, or metastatic niche formation in the subject.

In one aspect, a method of identifying a population of PM-LC distinguishable from PMN-MDSC in a subject involves obtaining a biological sample from the subject and identifying PM-LCs by detecting genes or pathways identified herein and/or detecting cell surface expression of Glut1, CXCR1, CXCR2, CD15, CD14, CD11 b, CD33 and/or CD66b. In one embodiment, the method includes administering an effective amount of an agent that alters neutrophil migration to reduce or inhibit tumor cell seeding, tumor metastasis, or metastatic niche formation in the subject.

In another aspect, a method of predicting the likelihood of metastasis in a subject who has cancer comprises obtaining a sample from the subject and assessing the sample for the presence of the PM-LC by detecting genes or pathways identified herein and/or detecting cell surface expression of Glut1, CXCR1, CXCR2, CD15, CD14, CD11b, CD33 and/or CD66b. In one embodiment, the method includes administering an effective amount of an agent that alters neutrophil migration to reduce or inhibit tumor cell seeding, tumor metastasis, or metastatic niche formation in the subject.

In another aspect, a method of decreasing metastasis and/or neutrophil migration in a subject who has cancer comprises obtaining a sample from the subject and assessing the sample for the presence of the PM-LC by detecting genes or pathways identified herein and/or detecting cell surface expression of Glut1, CXCR1, CXCR2, CD15, CD14, CD11b, CD33 and/or CD66b. In one embodiment, the method includes administering an effective amount of an agent that alters neutrophil migration to reduce or inhibit tumor cell seeding, tumor metastasis, or metastatic niche formation in the subject.

In another aspect, a method of determining a treatment regimen for a subject having cancer comprises obtaining a sample from the subject and assessing the sample for the presence of PM-LC by detecting genes or pathways identified herein and/or detecting cell surface expression of Glut1, CXCR1, CXCR2, CD15, CD14, CD11b, CD33 and/or CD66b. In one embodiment, the method includes administering an effective amount of an agent that alters neutrophil migration to reduce or inhibit tumor cell seeding, tumor metastasis, or metastatic niche formation in the subject.

In another aspect, a method of providing a personalized treatment regimen for a subject with cancer comprises determining whether the cancer has metastasized, and, if no metastasis is detected, determining whether PM-LC cells are present in a sample obtained from the patient by detecting genes or pathways identified herein and/or detecting cell surface expression of Glut1, CXCR1, CXCR2, CD15, CD14, CD11b, CD33 and/or CD66b. In one embodiment, when PM-LC are present, the method includes administering an effective amount of an agent that alters neutrophil migration to reduce or inhibit tumor cell seeding, tumor metastasis, or metastatic niche formation in the subject.

In one aspect, the method comprises detecting levels of one or more of the following: expression of Glut1, oxidative phosphorylation, glucose uptake, TCA cycle flux, glycolysis, ATP production, motility or migration, spontaneous migration, cytoplasmic ROS, the ER stress response, antigen-specific T cell suppression, myosin light chain 2 phosphorylation, and chemotaxis in response to CXCL8.

In another aspect, the method comprises detection of P2Y1 or P2Y2 receptor to identify PM-LC in a sample.

In a further aspect, PM-LCs are identified by increased levels of spontaneous migration.

In another aspect, a method is provided which comprises a treatment regimen that includes administering an effective amount of an agent that alters neutrophil migration to reduce or inhibit tumor cell seeding, tumor metastasis, or metastatic niche formation in a subject.

In yet another aspect, the method includes administering an agent that binds or inhibits a chemokine, chemokine receptor, or chemoattractant. The chemokine receptor may be CXCR1 of CXCR2. In certain embodiments, the agent binds or inhibits fMLP, CXCL8, or CXCL1.

In another aspect, the method comprises administering an agent that is an inhibitor or antagonist of pannexin-1 or connexin hemichannels. Suitable agents include peptide mimetics, nucleic acid molecules, small molecule compounds, antibodies, and derivatives thereof.

In another aspect, an agent that alters glycolysis is administered to a subject. In one aspect, the method comprises administering an agent that blocks or inhibits Glut1 function.

In yet another aspect, the method comprises delivering an agent that blocks or inhibits a purinergic receptor. The purinergic receptor may be P2X₁, P2Y₁, or P2Y₂ and the agent may be one or more of suramin, NF449, MRS2179, AR-C118925, BMS-884775, and GLS-409, or a salt or derivative thereof.

In one aspect, the method comprises administering an agent that reduces neutrophil ATP production or signaling. The agent may be apyrase.

In yet another aspect, the method comprises administering an agent that reduces or inhibits the ER stress response. The agent may be a small molecule modulator of B-I09 or one or more of CDN-1163, SERCA2b, TDI-194, AC-915, RX-1, ADoPep-2, and AC-2010, or a salt or derivative thereof. In yet another embodiment, the method comprises administering an antagonist or inhibitor of the expression, activity or activation of one or more of sXBP1, DDIT3 (CHOP), ATF4, ATF3, SEC61A, ARGI or NOS-2.

In a further aspect, the method comprises administering an agent to a tumor-bearing or non-tumor bearing tissue.

Other aspects and advantages of these compositions and methods are described further in the following detailed description of the preferred embodiments thereof.

### BRIEF DESCRIPTION OF THE DRAWINGS

FIG. 1A-FIG. 1C. Neutrophils from the bone marrow of three genetically engineered mouse models exhibit increased spontaneous migration. (FIG. 1A) Transwell assays evaluating the ability of neutrophils to migrate in response to CXCL1 and fMLP stimuli in indicated tumor models. Fold increase is calculated as the ratio of the number of cells that migrate in response to stimulus to the number of cells that spontaneously migrate. For RET model n=3, KPC model n=6, TRAMP model n=3. Mean and SD are shown. P values calculated between groups in two-sided Student's and are presented as range due to overcrowded figure. *−p<0.05; **p<0.01. (FIG. 1B) Spontaneous migration of neutrophils in indicated tumor models. For RET model n=5, for KPC model n=7, for TRAMP model n=5. (FIG. 1C) CXCR1 and CXCR2 expression on the cell surface of neutrophils. RET (n=5), TRAMP (n=4), KPC (n=6). *−p<0.05; **p<0.01 for significant differences between control and tumor-bearing mice. Statistics were calculated in two-sided Student's t-test.

FIG. 2A-FIG. 2G. Neutrophils from genetically engineered mouse models, but not transplantable tumor mouse models, exhibit an increased spontaneous migration. (FIG. 2A) Transwell assays evaluating the ability of peripheral blood neutrophils from RET melanoma mice to spontaneously migrate (left) and chemotax in response to CXCL1 (right). N=3, mean, and SD are shown. (FIG. 2B) Transwell assays evaluating the ability of neutrophils to spontaneously migrate (left) and chemotax in response to CXCL1 (right). N=3, mean, and SD are shown. (FIG. 2C-FIG. 2G) Analysis of neutrophil migration with time-lapse video. (FIG. 2C) Representative cell traces of neutrophils. Axes-400 μm. (FIG. 2D) The cell traces and mean-squared displacements of neutrophil migration. (FIG. 2E) speed, (FIG. 2F) persistence times, (FIG. 2G) random motility coefficients of BM neutrophils isolated from control tumor-free (n=5), EL4 (n=5) and RET melanoma (n=3) bearing mice. Mean and SD are shown. *−p<0.05; **p<0.01 in two-tailed Student's t-test.

FIG. 3A-FIG. 3G. Neutrophils from the early, but not late stages, of an orthotopic lung cancer model exhibit increased spontaneous migration. (FIG. 3A) Transwell assays evaluating the ability of BM neutrophils from mice bearing s.c. 4662 tumors derived from KPC mice to spontaneously migrate (left) and chemotaxis in response to CXCL1 (middle) or to fMPL (right). Mean, and SD are shown (n=4). P values are calculated in two-sided Student's t-test (not shown due to lack of significance). (FIG. 3B) Transwell assays evaluating the ability of neutrophils to spontaneously migrate. In one week LL2 experiments, n=5 for control group and n=6 for LL2 mice. In three week LL2 experiments, n=3 for control mice and n=4 for LL2 mice. Mean, and SD are shown. (FIG. 3C) Transwell assays of neutrophil chemotaxis in response to CXCL1. Mean and SD are shown. (FIG. 3D, FIG. 3E) Flow cytometry analysis of CD45.2⁺BM neutrophils from control mice and CD45.1⁺ neutrophils from one-week or three-week LL2 TB mice to migrate into the spleen and lung 1 hour after injection to naive CD45.1⁺CD45.2⁺ recipients. (FIG. 3D) Flow cytometry gating strategy with representative result from 6 performed experiments, (FIG. 3E) Ratio between CD45.1⁺ and CD45.2⁺ CD11b⁺Ly6G⁺Ly6C^{lo} neutrophils. In each experiment, the ratio of CD45.1⁺/CD45.2⁺ cells injected into recipient mice was set as the baseline=1. For one week LL2 experiments n=8, for three-week LL2 experiments n=6. Mean, and SD are shown. P values are calculated in two-sided Student's t-test from baseline within each group of LL2 mice and between the two groups of LL2 mice are shown. (FIG. 3F) Transwell migration assay of BM neutrophils from control and KPC mice with PanIn and invasive PDA. N=5 (control), n=5 (PanIn), n=3 (PDA). Mean and SD are shown. (FIG. 3G) The mean-squared displacements of neutrophil migration. Each curve represents cumulative results of traces 45-60 individual cells. In FIG. 3A-FIG. 3C, FIG. 3E, and FIG. 3F p values were calculated in two-sided Student's t-test. *−p<0.05, **−p<0.01. In FIG. 3G p values were calculated in two-way ANOVA test with Bonferroni correction for multiple comparisons. ***−p<0.0001 Two experiments with similar results were performed.

FIG. 4A-FIG. 4C. Transcriptome and functional activity of neutrophils in TB mice. BM Neutrophils were isolated from naive, one week and three-week LL2 TB mice. Gene expression profile was evaluated using RNA-seq. Three samples from each group of mice were evaluated. (FIG. 4A) Total number of changed genes, (FIG. 4B) significant (FDR<5%) changes in pathways between three-week and one-week TB mice. (FIG. 4C) Significant (FDR<5%) changes in regulators between three-week and one-week TB mice.

FIG. 5A-FIG. 5E. Suppressive activity of BM neutrophils in TB mice. (FIG. 5A) Antigen-specific proliferation of CD8⁺ T cells in the presence of BM Ly6G⁺ cells isolated from control mice and one or three weeks LL2 mice. Transgenic, Pmel splenocytes stimulated with specific peptide were used as responders. Proliferation was measured in triplicate by ³H thymidine uptake. Baseline of T cell proliferation in the absence of added neutrophils was set as 100%. Control −n=7, week 1 LL2 −n=10, week 3 LL2 −n=11. Mean, and SD are shown. (FIG. 5B) Antigen-specific proliferation of CD8⁺ T cells in the presence of BM Ly6G⁺ cells from naive mice, RET melanoma mice (left panel), LLC, or EL4 (right panel) mice. Pmel splenocytes stimulated with specific peptide were used as responders. Proliferation was measured in triplicate by ³H thymidine uptake. One experiment is shown. Three experiments with the same results were performed. Each experiment included 3 mice. Mean and SD are shown. (FIG. 5C) Suppressive activity of PMN from BM of KPC and TRAMP transgenic mice. OT-1 splenocytes stimulated with specific peptide were used as responders. Proliferation was measured in triplicate by ³H thymidine uptake. For KPC mice 5 experiments with similar results were performed. In TRAMP mice two experiments with the same results were performed. Mean and SD are shown. (FIG. 5D) Suppressive activity of neutrophils from KPC mice with PanIN and PDA. Proliferation was evaluated by ³H-thymidine uptake in triplicates. Means and SD of cumulative result from two mice are shown. (FIG. 5E) Cytokine protein expression array in sera of one- and three-week orthotopic LL2 TB mice. Experiments were performed in duplicates and individual data are shown. On the left cytokines with more than 2SD increase in sera of three-week LL2 mice over one-week LL2 mice. On the right cytokines with less than 2SD increase or decrease in three-week LL2 mice. * −p<0.05; **−p<0.01 in two-sided Student's t-test.

FIG. 6A-FIG. 6H. PM-LC have increased metabolic flux through oxidative phosphorylation and glycolysis and have more ATP than control neutrophils. (FIG. 6A and FIG. D) Oxygen consumption rate (OCR) and extracellular acidification rate (ECAR) of neutrophils from control, one-week, and three-week LL2 TB mice. After 3 basal measurements, cells were treated with oligomycin, FCCP, and rotenone and antimycin. (FIG. 6A) 18 measurements from 3 mice in each group were taken (left). Basal metabolic rates from 3 mice (right). (FIG. 6B) Spare respiratory capacity (maximal rate post-FCCP treatment—basal rate) of PM-LC from 3 control and RET melanoma mice. (FIG. 6C) Basal OCR of neutrophils from 3 control and 3 EL4 mice. (FIG. 6D) ECAR of neutrophils from 3 control, 3 one week LL2 and 3 three week LL3 mice. (FIG. 6E and FIG. 6F) Basal glycolytic rate of PM-LC from 3 control and 3 RET melanoma mice (FIG. 6E) and from 3 EL4 mice (FIG. 6F). Individual results and mean and SD are shown. P values were calculated in two-sided Student's t-test. *−p<0.05; **−p<0.01. (FIG. 6G) Ex vivo tracing of ¹³C₆-glucose metabolism in control neutrophils and PM-LC isolated from BM of one-week LL2 TB mice as determined by LC-MS. ¹³C labeling of pyruvate and lactate (M+3 for both) was used as a readout of glycolytic flux while labeling of citrate (M+2) was used as a readout of glucose flux into the TCA cycle. Changes in the isotopologue distribution (i.e. ¹³C labeling pattern) of the metabolites was analyzed using a grouped analysis, regular two-way ANOVA (Tukey correction) with multiple comparisons. * −p<0.05. Data are presented as mean peak areas and SD and are normalized to protein content. (n=3 individual mice per group). M+0, M+1, M+2, etc. indicate the number of ¹³C atoms in each metabolite. Black arrows indicate the flow of carbon. (FIG. 6H) LC-MS based measurement of intracellular ATP levels in control neutrophils and PM-LC. ATP levels were analyzed using an unpaired, two-tailed Student t-test. Data are presented as mean peak areas ±SD and are normalized to protein content. (n=3). P values were calculated in two-sided Student's t-test. **−p <0.01

FIG. 7A-FIG. 7H. PM-LC spontaneous migration is dependent upon pannexin-1 hemichannels, extracellular ATP, and P2X and P2Y receptors. (FIG. 7A) Transwell assays evaluating the ability of PM-LC from one-week LL2 mice (left) (n=3) and RET mice (right) (n=5) to spontaneously migrate upon pannexin-1 hemichannel inhibition (panx). (FIG. 7B) Transwell assays evaluating the ability of neutrophils from control mice (n=8) and PM-LC from one-week LL2 mice (n=10) to spontaneously migrate upon apyrase treatment. (FIG. 7C-FIG. 7F) Transwell assays evaluating the ability of neutrophils from control mice (n=6) and PM-LC from one-week LL2 mice (n=7) to spontaneously migrate upon pan P2R inhibitor, P2XR, P2X1, P2Y, and A3R inhibition with suramin, NF449, MRS2179, AR-C118925XX, and MRS 1191, respectively. In addition, the ability of PM-LC from RET melanoma mice (n=3) to spontaneously migrate was also evaluated in the presence of P2Y inhibitors MRS2179 and AR-C118925XX. Individual values and mean and SD are shown. (FIG. 7G) Migration of BM neutrophils from control mice (n=3) and PMN-MDSC from 3-week LL2 mice (n=3) in response to stimulation with different concentrations of ADP. Mean and SD are shown. P values are calculated in two-sided Student's t-test. *−p<0.05; **−p<0.01; ***−p<0.001 (FIG. 7H) pMLC2 in BM neutrophils isolated from naive mice and one-week LL2 TB mice. Experiments were performed three times with similar results.

FIG. 8A-FIG. 8H. Neutrophil migration in cancer patients. Spontaneous (n=24 for both healthy donors and cancer patients groups) (FIG. 8A) or chemokine induced (for healthy donor n=24 for media, n=10 for CXCL8 stimulation, n=6 for fMLP stimulation; for cancer patients n=14 for media, n=3 for CXCL8, and n=3 for fMLP); (FIG. 8B) migration of neutrophils from healthy individuals and cancer subjects. Individual results for each subject, mean and SD are shown. (FIG. 8C) CXCR1 and CXCR2 expression in neutrophils from healthy individual (n=7) and cancer patients (n=4 for CXCR1 and n=6 for CXCR2) assessed by flow cytometry. Individual results for each subject, mean and SD are shown. (FIG. 8D and FIG. 8E) Spontaneous (n=12) (FIG. 8D) and CXCL1 stimulated (n=11 for PMN and n=13 for PMN-MDSC) (FIG. 8E) migration of neutrophils and PMN-MDSC from the same cancer patients. (FIG. 8F) Expression of chemokine receptors in human neutrophils. Indicated chemokine receptors were measured by flow cytometry on PMN-MDSC and neutrophils from cancer patients. N=4 for CXCR1 and n=6 for CXCR2. (FIG. 8G) Percentage of CD45⁻CFSE⁺ LLC cells in lungs from naive mice 12 hours after intravenous injection. BM Ly6G⁺ neutrophils (PMN) from naïve (n=7), one week (n=5), or three-week (n=5) LL2 mice were injected 6 hours before tumor cells. (FIG. 8H) IVIS-based analysis of ex-vivo luciferase activity in excised lungs from naive mice, which were injected i.v. with indicated BM Ly6G⁺ cells 5 hours before the i.v. injection of LL2 tumor cells. N=3. Detection was performed two weeks after the injection. In all panels mean and SD are shown. P values are calculated in two-sided Student's t-test. * −p<0.05; **−p<0.01; ***−p<0.001; ****−p<0.0001.

FIG. 9A-FIG. 9D. The total number of CD11b⁺Ly6C^{low}Ly6G⁺ neutrophils in BM of tumor-bearing mice. (FIG. 9A) GEM models (n=6 for RET mice, n=5 for TRAMP mice, n=3 for KPC mice), (FIG. 9B) ectotopic s.c. models (n=5 for EL4 mice), (FIG. 9C) Example of luciferase activity in lungs extracted from mice injected i.v. with 5×10⁴ LL2 tumor cells. (FIG. 9D) orthotopic LL2 model (n=3). Individual results, mean and SD are shown. P values were calculated in two-sided Student's t-test.

FIG. 10. Network Ingenuity Pathway Analysis of genes changed at week 1 compared to naive. Top network (score=53) annotated with Top Diseases and Functions as “Energy Production, Nucleic Acid Metabolism, Small Molecule Biochemistry” is shown.

FIG. 11A-FIG. 11B. Top 30 differentially expressed known genes. (FIG. 11A) At week 1 vs Control. (FIG. 11B) At week 3 vs Control. Insignificant fold changes with nominal p>0.05 are shown in white.

FIG. 12A-FIG. 12D. Cytoplasmic ROS level in neutrophils. Spontaneous and stimulated (TBHP) ROS level in BM neutrophils from naïve (n=7), one- (n=4) and three-week (n=6) LL2 mice (FIG. 12A) RET melanoma (n=3) (FIG. 12B) EL4 (n=3) and LLC (n=3) mice (FIG. 12C) Individual results for each mouse, mean and SD are shown. P values in two-sided Student's t-test are shown. (FIG. 12D) Mitochondrial mass of neutrophils and PMN-MDSC. Mitochondrial mass was measured using MitoTracker™ Green-FM and flow cytometry in BM neutrophils from indicated tumor models. KPC (n=6), RET (n=3), TRAMP (n=6), CT26 (n=3), LLC (n=3), EL4 (n=5). Individual results for each mouse, mean and SD are shown.

FIG. 13. Illustration of the flow of ¹³C₆-glucose through glycolysis and into the TCA cycle. The black circles represent carbon-13, the clear circle represent carbon-12. The six carbons of glucose are broken into two molecules of three carbons each half through glycolysis. Hence, the appearance of pyruvate containing three carbon-13 atoms (i.e. M+3) is a readout of glycolysis. Subsequently, if pyruvate is converted to lactate, the lactate will also have all three carbons labeled (M+3). However, if pyruvate enters the mitochondria, pyruvate dehydrogenase catalyzes its decarboxylation (loss of CO₂) thereby creating a two carbon-13 labeled acetyl-CoA molecule. The two carbons of acetyl-CoA are then used in the synthesis of citrate (M+2) and can be used as a readout of glucose flux into the TCA cycle.

FIG. 14A-FIG. 14D. Glucose metabolism in neutrophils. (FIG. 14A) Flow of ¹³C6-glucose through glycolysis in BM neutrophils from control and TB mice with 3-week s.c. LL2 tumor (n=3). (FIG. 14B) ATP level in neutrophils (n=3) (FIG. 14C) Expression of glucose transporter on the surface of neutrophils from naïve and one-week LL2 TB mice (n=7). (FIG. 14D) Expression of Glut1 on the surface of neutrophils from naïve and three-week LL2 TB mice (n=3). Individual results for each mouse, mean and SD are shown. Significant p values in two-sided Student's t-test are shown.

FIG. 15A-FIG. 15E. Expression of F-actin and genes associated with the glycolytic pathway. (FIG. 15A) Expression of indicated genes in neutrophils from control and RET melanoma mice measured in real-time quantitative PCR (n=3). Individual results for each mouse, mean and SD are shown. (FIG. 15B) F-actin was measured using flow cytometry before and after stimulation with CXCL1 or fMLP in BM neutrophils from indicated tumor models. Typical example of 4 experiments is shown. (FIG. 15C) F-actin in unstimulated cells evaluated by flow cytometry. Individual results for each mouse, mean and SD are shown. (FIG. 15D) F-actin in neutrophils and PMN-MDSC measured by flow cytometry (n=4). Individual results for each mouse, mean and SD are shown. Significant p values in two-sided Student's t-test are shown. (FIG. 15E) pMLC2 in neutrophils from control tumor-free and one-week LLC TB mice. Neutrophils were treated with vehicle alone or with P2Y2 inhibitor (ARC) for 1 hour before Western blot analysis. Two experiments with similar results were performed.

FIG. 16. Model of PM-LC involvement in tumor progression. During early stages of tumor development, BM neutrophils are characterized by ER stress, and increased OXPHOS and glycolysis, This results in increased spontaneous motility mediated by ATP and purinergic receptors in autocrine and paracrine fashion. These cells actively migrate to uninvolved tissues and promoted tumor cell seeding and formation of metastasis. At later stages, when tumor burden is bigger and tumors are infiltrated with immune cells or contain necrotic loci with associated inflammation, it results in generation of PMN-MDSC, characterized by immune suppressive activity, activation of pro-inflammatory and oxidative damage signaling. These cells actively migrate to the sites with established tumors (primary of metastatic) and promote further tumor growth.

### DETAILED DESCRIPTION OF THE INVENTION

Although neutrophils have been linked to the formation of the pre-metastatic niche, the mechanism of their migration to distant uninvolved tissues has remained elusive. As disclosed herein, bone marrow neutrophils from mice with early-stage cancers exhibit higher levels of more spontaneous migration to tissues. These cells lack immunosuppressive activity but have elevated rates of oxidative phosphorylation and glycolysis, and much more production of ATP.

Their enhanced spontaneous migration is mediated by the binding of ATP to purinergic receptors. In ectopic tumor models and the late stages of cancers, bone marrow neutrophils exhibit potent immunosuppressive activity. However, these cells have metabolic and migratory activity indistinguishable from that of control neutrophils. A similar pattern of migration can be found in neutrophils and polymorphonuclear myeloid-derived suppressor cells from patients with cancer. Thus, the dynamic changes that neutrophils undergo in patients with cancer can contribute to early tumor dissemination.

The findings described herein identifying specific populations of neutrophils that are distinguishable from PMN-MDSC in terms of their phenotype and functional characteristics. These findings are the basis for novel diagnostic and therapeutic methods and compositions for the treatment of cancer.

Unless defined otherwise in this specification, technical and scientific terms used herein have the same meaning as commonly understood by one of ordinary skill in the fields of biology, biotechnology and molecular biology and by reference to published texts, which provide one skilled in the art with a general guide to many of the terms used in the present application. The definitions herein are provided for clarity only and are not intended to limit the claimed invention.

“Patient” or “subject” as used herein means a mammalian animal, including a human, a veterinary or farm animal, a domestic animal or pet, and animals normally used for clinical research. In one embodiment, the subject of these methods and compositions is a human. In one embodiment, the subject is a patient who has been diagnosed with cancer. In another embodiment, the subject has not yet received a diagnosis of metastatic cancer. In another embodiment, the subject has received a diagnosis of metastatic cancer.

The term “cancer” or “tumor” as used herein refers to, without limitation, refers to or describes the physiological condition in mammals that is typically characterized by unregulated cell growth. By cancer, as used herein, is meant any form of cancer, including hematological cancers, e.g., leukemia, lymphoma, myeloma, bone marrow cancer, and epithelial cancers, including, without limitation, breast cancer, lung cancer, prostate cancer, colorectal cancer, brain cancer, endometrial cancer, esophageal cancer, stomach cancer, bladder cancer, kidney cancer, pancreatic cancer, cervical cancer, head and neck cancer, ovarian cancer, melanoma, leukemia, myeloma, lymphoma, glioma, Non-Hodgkin's lymphoma, leukemia, multiple myeloma, and multidrug resistant cancer. In one embodiment, the cancer is a solid tumor.

A “tumor” is an abnormal mass of tissue that results from excessive cell division that is uncontrolled and progressive, and is also referred to as a neoplasm. The term “tumor,” as used herein, refers to all neoplastic cell growth and proliferation, whether malignant or benign, and all pre-cancerous and cancerous cells and tissues. Whenever the term “lung cancer” is used herein, it is used as a representative cancer for demonstration of the use of the methods and compositions described herein.

As used herein, the term “treatment of cancer” or “treating cancer” can be described by a number of different parameters including, but not limited to, reduction in the size of a tumor in an animal having cancer, reduction in the growth or proliferation of a tumor in an animal having cancer, preventing, inhibiting, or reducing the extent of metastasis, and/or extending the survival of an animal having cancer compared to control. In certain embodiments, treatment results in a reduced risk of distant recurrence or metastasis in a subject.

The term “biological sample” as used herein means any fluid or suspension or tissue from a subject, including samples that contain PMN, PMN-MDSCs, or MDSC-like neutrophils (also called PM-LC). The sample in certain embodiments contains cells that are both PMN-MDSC and MDSC-like neutrophils. The sample in one embodiment contains cells with cell surface antigens indicative of polymorphonuclear cells or neutrophils. In one embodiment, cells (neutrophils) in the sample express CD66b⁺. In another embodiment, cells (neutrophils) in the sample express CD15⁺ In still another embodiment, neutrophils in the sample express one or more of CD11b, CD33, CD15, CD14, CD11b, CD33 and CD66b. The most suitable samples for use in the methods and with the diagnostic compositions or reagents described herein are samples or suspensions which require minimal invasion for testing, e.g., blood samples, including whole blood, and any samples containing shed or circulating tumor cells. It is anticipated that other biological samples that contain cells at a sufficiently detectable concentration, such as peripheral blood, serum, saliva or urine, vaginal or cervical secretions, and ascites fluids or peritoneal fluid may be similarly evaluated by the methods described herein. In one embodiment, the sample is a tumor secretome, i.e., any fluid or medium containing the proteins secreted from the tumor. These shed proteins may be unassociated, associated with other biological molecules, or enclosed in a lipid membrane such as an exosome. Also, circulating tumor cells or fluids or tissues containing them are also suitable samples for evaluation in certain embodiments of this invention. In another embodiment, the biological sample is a tissue or tissue extract, e.g., biopsied material, containing the PMN-MDSC or MDSC-like neutrophils. In certain embodiments, the biopsied material is obtained from a patient lymph node, lung, or spleen. In one embodiment, such samples may further be diluted with or suspended in, saline, buffer or a physiologically acceptable diluent. Alternatively, such samples are tested neat. In another embodiment, the samples are concentrated by conventional means.

The term “ER stress response” as used herein refers to a response mediated by the endoplasmic reticulum to protect cells from various stress conditions including hypoxia, nutrient deprivation, low pH, etc. and includes three major signaling cascades initiated by three protein sensors: PERK (protein kinase RNA (PKR)-like ER kinase), IRE-1 (inositol-requiring enzyme 1) and ATF6 (activating transcription factor 6) (See, for example, Holcik, M et al., Translational control in stress and apoptosis. Nature Rev Mol Cell Biol, April 2005. 6: 318, which is incorporated herein by reference). Antagonists or inhibitors of ER stress include ligands, e.g., antibodies, fragments thereof, small molecules that can block the activity, function or activation of the regulators of ER stress, identified herein.

The term “myeloid-derived suppressor cells” or “MDSCs” refers to cells of myeloid origin that are undergo expansion in a cancer-related context and have been described as having certain immune-suppressive functions. MDSC comprise two major subsets termed polymorphonuclear (PMN) and monocytic (M)-MDSC, also PMN-MDSC and M-MDSC, respectively. PMN-MDSC and M-MDSC are phenotypically and morphologically distinct, but share some overlapping functional characteristics and biochemical traits. In certain embodiments, PMN-MDSCs and M-MDSCs are identified or separated using markers including, for example, CD11b, CD14, CD15, CD66, HLA-DR, CD33. In human peripheral blood, for example, PMN-MDSC can be identified as CD11b⁺CD14⁻CD15⁺ or CD11b⁺CD14⁻CD66b⁺ and M-MDSC as CD11b⁺CD14⁺HLA-DR^{-/lo}CD15⁻. In certain embodiments, lectin-type oxidized LDL receptor 1 (LOX-1) is used as a marker to identify PMN-MDSCs in a sample. In certain embodiments, LOX-1 expression distinguishes PMN-MDSCs from, for example, neutrophils or PM-LCs in a sample. In some embodiments, PMN-MDSCs are identified based on the expression or level of expression of certain genes that make up a gene signature, which may include one or more of HLA-DPA1, HLA-DRA, EBI2, OLR1 THBS1, CD36, MMD, ASGR1, CD69, HLA-DRB3, CD74, RPSA, HLA-DQA1, CD86, PTGER2, ITGB5, CD79B, CD79A, IL10RA, PLXNB2, ITGB1, PLAUR, CD247, SCARB2, CD1D, GPBAR1, CLEC1B, TFRC, ITGB3, CD300C, ITGA22B and CXCR5. In certain embodiments, MDSCs are identified based on expression of one or more of MYCN, CSF3, IL3, TGFβ1, TNF, LDL, RAF1, APP, IL6 PDGFBB, EPO, CD4OLG, Nek, IL13, AGT, IL1β, ERBB2, MAP2K1, VEGFα, CSF1, FLI1, or Fin, CD15, CD66b or CD33. Suitable markers and functional assays for identifying or distinguishing MDSC subsets are described in the art (See, for example, Bronte, V. et al. Recommendations for myeloid-derived suppressor cell nomenclature and characterization standards. Nat. Commun. 7: 12150 (2016), which is incorporate herein by reference).

The term “PMN-MDSC-like cells” or “PM-LC” as used herein refers to myeloid cells that are have features or functions that distinguish them from PMN-MDSC. As described herein, the present inventors have identified phenotypic markers, genes, and functional characteristics that identify and/or distinguish PM-LCs from populations of neutrophils, M-MDSCs, and/or PMN-MDSCs. For example, PM-LCs lack immunosuppressive activity that is typical of PMN-MDSC. In some cases, for example, PM-LC lack the ability to suppress antigen specific T cell responses or not as effective as PMN-MDSC to suppress antigen-specific T cell responses. PM-LC may also exhibit one or more of increased level of Glut1 expression, a higher oxygen consumption rate, including increased oxidative phosphorylation (OXPHOS), increased glucose uptake, increased TCA cycle flux, glycolysis, increased ATP production, increased motility or migration, increased spontaneous migration, increased ER stress response, increased myosin light chain 2 phosphorylation, and increased chemotaxis in response to CXCL8.

As used herein for the described methods and compositions, the term “antibody” refers to an intact immunoglobulin having two light and two heavy chains or fragments thereof capable of binding to a biomarker protein or a fragment of a biomarker protein. Thus, a single isolated antibody or an antigen-binding fragment thereof may be a monoclonal antibody, a synthetic antibody, a recombinant antibody, a chimeric antibody, a humanized antibody, a human antibody, or a bi-specific antibody or multi-specific construct that can bind two or more antigens.

The term “antibody fragment” as used herein for the described methods and compositions refers to less than an intact antibody structure having antigen-binding ability. Such fragments, include, without limitation, an isolated single antibody chain or an scFv fragment, which is a recombinant molecule in which the variable regions of light and heavy immunoglobulin chains encoding antigen-binding domains are engineered into a single polypeptide. Other scFV constructs include diabodies, i.e., paired scFvs or non-covalent dimers of scFvs that bind to one another through complementary regions to form bivalent molecules. Still other scFV constructs include complementary scFvs produced as a single chain (tandem scFvs) or bispecific tandem scFvs.

The terms “analog”, “modification” and “derivative” refer to biologically active derivatives of the reference molecule that retain desired activity as described herein. In general, the term “analog” refers to compounds having a native polypeptide sequence and structure with one or more amino acid additions, substitutions (generally conservative in nature) and/or deletions, relative to the native molecule, so long as the modifications do not destroy activity and which are “substantially homologous” to the reference molecule as defined herein. Preferably, the analog, modification or derivative has at least the same desired activity as the native molecule, although not necessarily at the same level. The terms also encompass purposeful mutations that are made to the reference molecule. Particularly preferred modifications include substitutions that are conservative in nature, i.e., those substitutions that take place within a family of amino acids that are related in their side chains. Specifically, amino acids are generally divided into four families: acidic, basic, non-polar and uncharged polar. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the molecule of interest may include up to about 5-20 conservative or non-conservative amino acid substitutions, so long as the desired function of the molecule remains intact. One of skill in the art can readily determine regions of the molecule of interest that can tolerate change by reference to Hopp/Woods and Kyte Doolittle plots, well known in the art.

By “change in expression” or “altered expression” or similar phrases is meant an upregulation in the expression level of a nucleic acid sequence, e.g., genes or transcript, or cell surface marker, in comparison to the selected reference standard or control; a downregulation in the expression level of a nucleic acid sequence, e.g., genes or transcript, or cell surface maker, in comparison to the selected reference standard or control; or a combination of a pattern or relative pattern of certain upregulated and/or down regulated genes and/or cell surface markers. The degree of change in expression can vary with each individual gene or marker, or for among subjects.

The terms “differentially expressed gene”, “differential expression” and their synonyms, which are used interchangeably, refer to a gene whose expression is activated to a higher or lower level in a subject suffering from a disease, specifically cancer, relative to its expression in a control subject, such as a healthy subject. The terms also include genes whose expression is activated to a higher or lower level at different stages of the same disease (e.g., a subject having non-metastatic vs. metastatic disease). It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects, non-health controls and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is a statistically significant (p<0.05) difference in gene expression between the subject and control samples.

Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

The terms “a” or “an” refers to one or more, for example, “a cell” is understood to represent one or more cells. As such, the terms “a” (or “an”), “one or more,” and “at least one” are used interchangeably herein.

The term “about” as used herein when referring to a measurable value such as an amount, a temporal duration, and the like, is meant to encompass variations of up to ±10% from the specified value; as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.”

The terms “compound”, “composition”, or “substance” as used herein may be used interchangeably to discuss the therapeutic composition.

### Methods

Provided herein, in some aspects, are various methods which identify the presence of PM-LC in a subject having cancer. In some embodiments, the methods include obtaining a biological sample from a subject; and assaying for the presence of PM-LC.

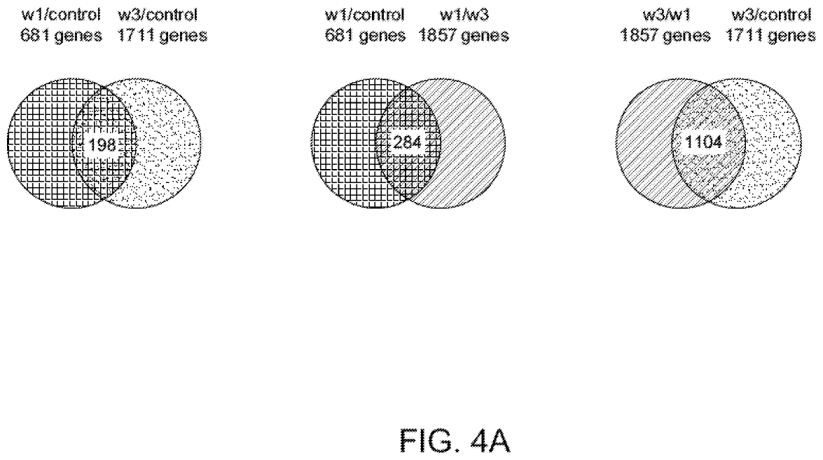

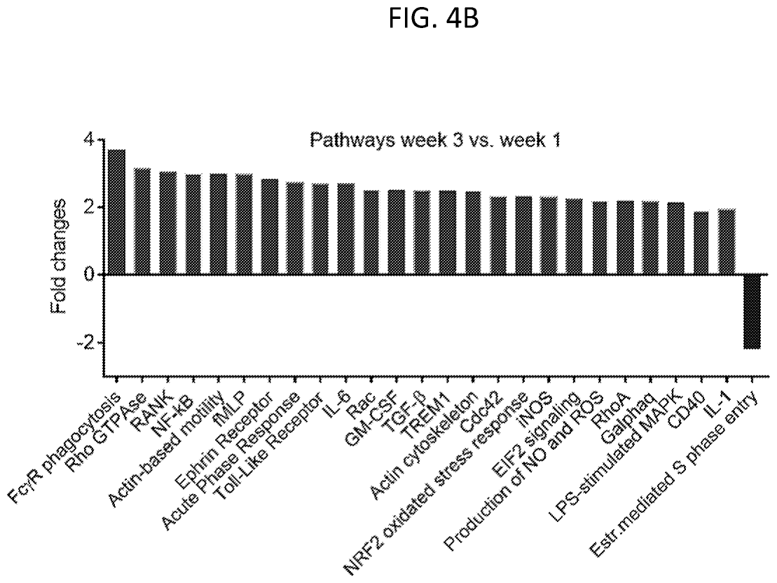

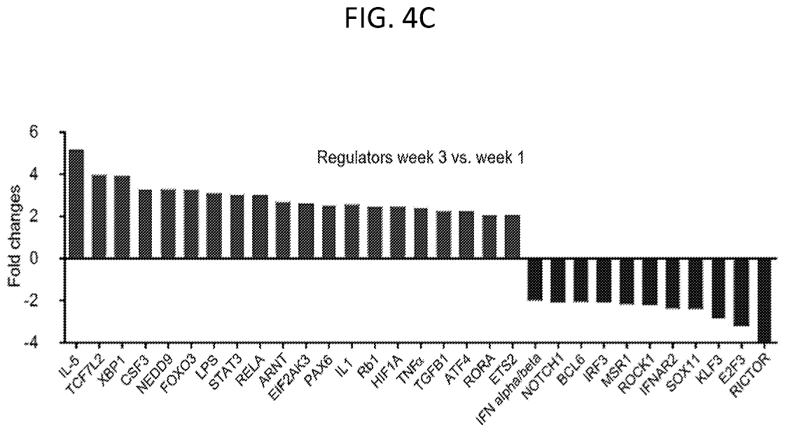

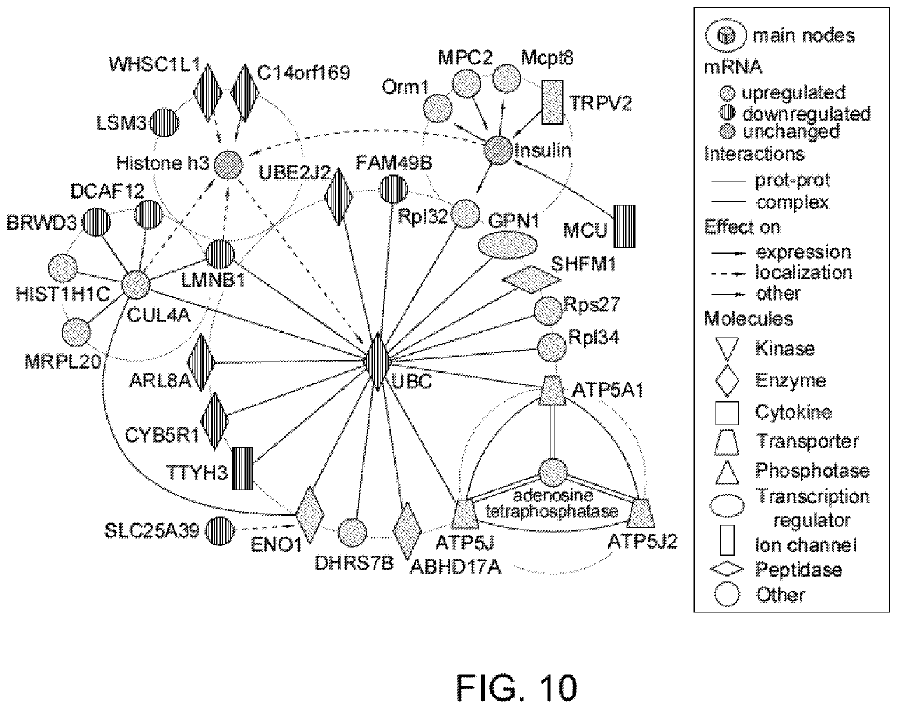

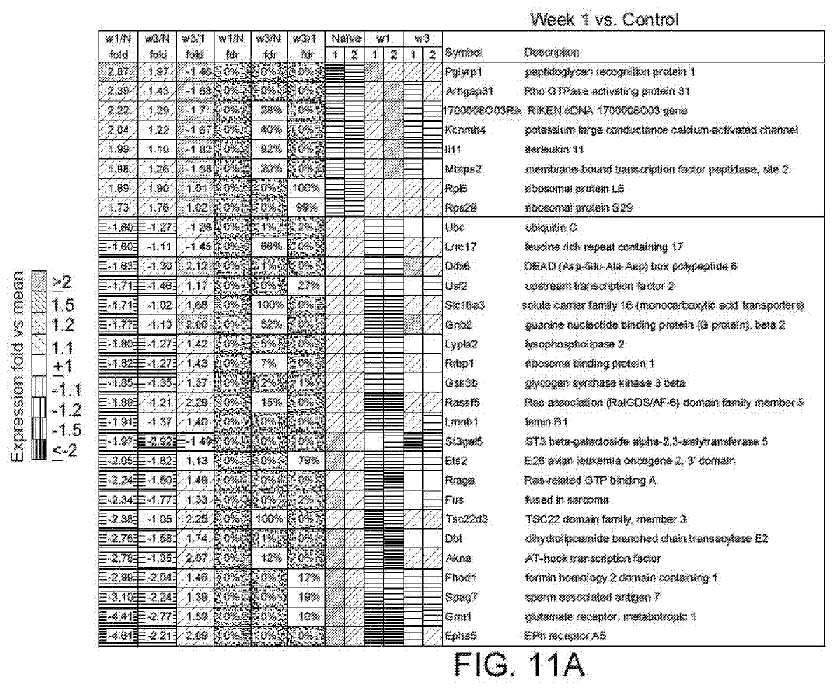

Detecting the presence of PM-LC includes one or more of the following steps: a) detecting one or more of genes or pathways identified in Table 1 or FIG. 4B, FIG. 4C, FIG. 10, FIG. 11A and FIG. 11B; b) detecting cell surface expression of one or more of Glut1, CXCR1, CXCR2, CD15, CD14, CD11b, CD33 and/or CD66b, and c) detecting one or more of levels of expression of Glut1, oxidative phosphorylation, glucose uptake, TCA cycle flux, glycolysis, ATP production, motility or migration, spontaneous migration, cytoplasmic ROS, the ER stress response, antigen-specific T cell suppression, myosin light chain 2 phosphorylation, and chemotaxis in response to CXCL8.

TABLE 1

| | | | Differentially | Differentially |
|---|---|---|---|---|
| Pathways | Regulators | Genes | expressed genes | expressed genes |
| (FIG. 4B) | (FIG. 4C) | (FIG. 10) | (FIG. 11A) | (FIG. 11B) |
| FcγR phagocytosis | IL-5 | WHSC1L1 | Pglyrp1 | BC100530 |
| Rho GTPase | TCF7L2 | C14orf169 | Arhgap31 | Stfa2 |
| RANK | XBP1 | LSM3 | 1700008O03Rik | Prok2 |
| NF-κB | CSF3 | Histone h3 | Kcnmb4 | Stfa3 |
| actin-based | NEDD9 | DCAF12 | Il11 | Fign12 |
| motility | | | | |
| fMLP | FOXO3 | BRWD3 | Mbtps2 | 0610040J01Rik |
| ephrin receptor | LPS | HIST1H1C | Rp16 | Cd38 |
| acute phase | STAT3 | LMNB1 | Rps29 | Cd244 |
| response | | | | |
| toll-like receptor | RELA | CUL4 | Ubc | Asprv1 |
| IL-6 | ARNT | MRPL20 | Lrrc17 | Stfal |
| Rac | EIF2AK3 | ARL8 | Ddx6 | Stfa2l1 |
| GM-CSF | PAX6 | CYB5R1 | Usf2 | Socs3 |
| TGF-β | IL1 | TTYH3 | Slc16a3 | 6330416G13Rik |
| TREM1 | Rb1 | SLC25A39 | Gnb2 | Il4ra |
| actin cytoskeleton | HIF1A | ENO1 | Lypla2 | Atp13a2 |
| cdc42 | TNFα | DHRS7B | Rrbp1 | Cd14 |
| NRF2 oxidated | TGFB1 | MPC2 | Gsk3b | Ifitm6 |
| stress response | | | | |
| iNOS | ATF4 | Orm1 | Rassf5 | Ctnnbip1 |
| EIF2 signaling | RORA | Mcpt8 | Lmnb1 | Zfp36l1 |
| Production of NO | ETS2 | TRPV2 | St3gal5 | Spata 13 |
| and ROS | | | | |
| RhoA | IFN | Insulin | Ets2 | Ddi2 |
| | alpha/beta | | | |
| Galphaq | NOTCH1 | UBE2J2 | Rraga | Tgfbi |
| LPS-stimulated | BCL6 | FAM49B | Fus | Snx1 |
| MAPK | | | | |
| CD40 | IRF3 | Rpl32 | Tsc22d3 | Emilin2 |
| IL-1 | MSR1 | GPN1 | Dbt | Ube2h |
| Estr. mediated S | ROCK1 | MCU | Akna | S100a6 |
| phase entry | | | | |
| | IFNAR2 | SHFM1 | Fhod1 | Atg1612 |
| | SOX11 | Rps27 | Spag7 | Il1rn |
| | KLF3 | Rpl34 | Grml | Acta2 |
| | E2F3 | UBC | Epha5 | Rasl11b |
| | RICTOR | ATP5A1 | | |
| | | ABHD17A | | |
| | | ATP5J | | |
| | | ATP5J2 | | |

In one embodiment, the subject has cancer selected from melanoma, prostate, clear cell renal cell carcinoma, breast cancer, other skin cancers, and any other cancers that can metastasize via the lymphatic system, including but not limited to lung, non-small cell lung, pancreatic, colorectal, head and neck, cervical, endometrial, testicular, and ovarian cancer. In one embodiment, the cancer is melanoma.

In some embodiments of the methods described herein, PM-LC are identified based differential expression of one or more genes or pathways identified in Table 1 or FIG. 4B, FIG. 4C, FIG. 10, FIG. 11A and FIG. 11B . Methods of identifying expression of genes are known in the art. See, e.g., WO 2017/233216, which is incorporated herein by reference. In one embodiment, the methods utilize one or more polynucleotides or oligonucleotides or ligands, wherein each polynucleotide or oligonucleotide or ligand hybridizes to a different gene, gene fragment, gene transcript or expression product in a sample selected from the genes of Table 1 or FIG. 4B, FIG. 4C, FIG. 10, FIG. 11A and FIG. 11B.

In one embodiment, at least one polynucleotide or oligonucleotide or ligand is attached to a detectable label. In certain embodiments, each polynucleotide or oligonucleotide is attached to a different detectable label, each capable of being detected independently.

In another embodiment, the composition comprises a capture oligonucleotide or ligand, which hybridizes to at least one polynucleotide or oligonucleotide or ligand. In one embodiment, such capture oligonucleotide or ligand may include a nucleic acid sequence which is specific for a portion of the oligonucleotide or polynucleotide or ligand which is specific for the gene of interest. The capture ligand may be a peptide or polypeptide which is specific for the ligand to the gene of interest. In one embodiment, the capture ligand is an antibody, as in a sandwich ELISA.

The capture oligonucleotide also includes a moiety which allows for binding with a substrate. Such substrate includes, without limitation, a plate, bead, slide, well, chip or chamber. In one embodiment, the composition includes a capture oligonucleotide for each different polynucleotide or oligonucleotide which is specific to a gene of interest. Each capture oligonucleotide may contain the same moiety which allows for binding with the same substrate. In one embodiment, the binding moiety is biotin.

In one aspect, a method of identifying PM-LCs is provided. The method includes obtaining a biological sample from a subject and identifying PM-LCs in the sample by detecting expression of genes or markers identified herein. PM-LC may also be identified based on cell surface expression of markers identified herein that distinguish PM-LC from other populations of cells in sample, for example PMN-MDSC. In certain embodiments, the differential expression of one or more genes or markers identifies the presence of or distinguishes a population of cells, e.g., PM-LCs, from another population of cells, such as PMN-MDSC, in the same subject or in the same sample, or in a sample obtained from a different subject, for example a heathy subject or subject with cancer. In certain aspects, a combination of gene expression and cell surface expression of phenotypic markers is used to identify PM-LCs in the sample or to distinguish PM-LCs from other cells in the sample.

For various cell surface markers described herein, methods for detection and measuring levels of expression, as well as marker-specific antibodies, are well known in the art. Suitable methods of detection include immunohistochemistry and immunofluorescent staining of tissue sections or cell suspensions obtained from a subject using labeled antibodies and reagents. Cell surface markers that are particularly useful for the methods described herein include markers that are expressed on populations of blood cells, including myeloid and myeloid-derived populations. In some instances, a patient sample is subjected to enrichment or purification. For example, in the case of tissue sample or biopsy, the sample maybe homogenized using enzymatic or mechanical means to produce a cell suspension. In the case of a peripheral blood sample, a Ficoll gradient or similar methods of gradient centrifugation may be utilized to fractionate the cells in the sample. Cells in a patient sample may also be enriched or purified using magnetic beads coated with, for example, antibodies that bind a surface antigen such as CD15.

The various markers described herein can be used to identify PMNs, MDSCs (including PMN-MDSCs and M-MDSCs), and PM-LCs. Markers and schemes suitable for identifying or distinguishing populations of PMN and PM-MDSCs are known in the art (see, e.g., Bronte, V. et al. Recommendations for myeloid-derived suppressor cell nomenclature and characterization standards. Nat. Commun. 7: 12150 (2016)). In certain embodiments, cells are identified using antibodies specific for one or more of CD15, CD14, CD11b, CD33 and/or CD66b. In certain embodiments, PM-LC are identified and/or distinguished from other cell populations, in particular PMN-MDSC, based on surface expression of purinergic receptors (such as P2X1, P2Y1, or P2Y2) or glucose transporters (for example, Glut1 and Glut4). In some embodiments, PM-LCs are identified based on differential expression of chemokine receptors, such as CXCR1 and CXCR1.

In another embodiment, PM-LC are identified by detecting one or more of levels of expression of Glut1, oxidative phosphorylation, glucose uptake, TCA cycle flux, glycolysis, ATP production, motility or migration, spontaneous migration, cytoplasmic ROS, the ER stress response, antigen-specific T cell suppression, myosin light chain 2 phosphorylation, and chemotaxis in response to CXCL8. Methods of assaying for these properties are known in the art, and exemplified herein.

In a further aspect, methods of predicting an increased likelihood of metastasis in a subject are provided. These methods include assaying for the presence of PM-LC in a biological sample. In one embodiment, the presence of PM-LC in the sample is indicative of an increased risk of metastasis as compared to a subject who does not have PM-LC present. In another embodiment, an increase in the number of PM-LC in the sample as compared to a control is indicative of an increased risk of metastasis as compared to a subject who has a lower number of PM-LC present.

In another aspect, a method of inhibiting or reducing metastasis, or the risk thereof, is provided. In one embodiment, the method includes assaying for the presence of PM-LC in a sample. Upon a finding of PM-LC, therapeutically effective amounts of one or more of the compositions described herein may be administered, or the treatment regiment altered.

The term “therapeutically effective amount” or “effective amount” refers to an amount agent that when administered alone or in combination with an additional therapeutic agent to a cell, tissue, or subject is effective to prevent or ameliorate the tumor-associated disease condition or the progression of the disease, e.g., metastasis. A therapeutically effective dose further refers to that amount of the compound sufficient to result reduction, prevention or inhibition of metastasis. For example, when in vivo administration of an agent is employed, normal dosage amounts may vary from about 10 ng/kg to up to 100 mg/kg of subject body weight or more per dosage or per day, preferably about 1 μg/kg to 50 mg/kg, optionally about 100 μg/kg to 20 mg/kg, 500 μg/kg to 10 mg/kg, or 1 mg/kg to 10 mg/kg, depending upon the route of administration.

A reduction or inhibition of metastasis can be measured relative to the incidence observed in the absence of the treatment and, in further testing, inhibits metastatic tumor growth. The tumor inhibition can be quantified using any convenient method of measurement. The incidence of metastasis can be assessed by examining relative dissemination (e.g., number of organ systems involved) and relative tumor burden in these sites. Metastatic growth can be ascertained by microscopic or macroscopic analysis, as appropriate. Tumor metastasis can be reduced by about 10%, 20%, 30%, 40%, 50%, 60%, 70%, 80%, 90%, 95% or greater.

Alternatively, a reduction or inhibition of metastasis is assessed based on the statistical risk of metastasis based on the type and stage of cancer, among other factors. Such risk may be assessed against a control patient or population. In one embodiment, the control patient or population is a healthy subject (or population thereof) or subject having a lesser stage of the same or similar cancer.

### Treatment Regimens

Also provided herein are methods of preventing or decreasing metastasis in a subject in need thereof. In one embodiment, the method includes administering an effective amount of an agent that alters neutrophil migration to reduce or inhibit tumor cell seeding, tumor metastasis, or metastatic niche formation in the subject. Such agents are described herein.

In another aspect, a method of treating cancer includes administering an effective amount of an agent that alters neutrophil migration to reduce or inhibit tumor cell seeding, tumor metastasis, or metastatic niche formation in the subject.

Also provided are methods of providing personalized treatment regimens for subjects having cancer. As described herein PM-LCs are shown to be present in potential metastatic sites even in non-metastatic subjects. Thus, in certain embodiments, the method includes determining whether the cancer has metastasized; and, if no metastasis is detected, determining whether PM-LC cells are present in a sample obtained from the patient. PM-LC cells may be screed for using any of the techniques described herein. In one embodiment, if the presence of PM-LC cells is detected, the treatment regimen is altered as compared to a treatment regimen for a non-metastatic stage of the same cancer type. In one embodiment, a metastatic treatment regimen is administered. In some embodiments, the altered treatment regimen includes administration of any of the compositions described herein, including purinergic receptors antagonists, inhibitors of pannexin-1, and ER stress antagonists.

Metastatic treatment regimens can be determined by the person of skill in the art based on the type and stage of cancer, and other factors (including presence/absence of biomarkers, etc). Metastatic treatment regimens are generally more aggressive than treatment regimens for non-metastatic cancer. Treatments for metastatic cancer include surgery, chemotherapy, hormone therapy, biologic therapy, and radiation therapy, and combinations thereof.

Methods of determining whether metastasis has occurred are known in the art and include, without limitation, computed tomography (CT or CAT) scan, Positron emission tomography (PET) or PET-CT scan, sentinel node biopsy, X-rays, bone scan, magnetic resonance imaging (MM), serum blood chemistry tests, complete blood count, and blood tumor marker tests.

Other therapeutic benefits or beneficial effects provided by the methods described herein may be objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumor or cancer, or metastasis. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore be a cure, such as destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

Various routes of administration are useful in these methods. In one embodiment, the recombinant protein or vector is delivered to the tumor site itself. In another embodiment, the recombinant protein, agonist or vector is delivered to tumor draining lymph node (TDLN) or nodes or other lymph node or nodes. Draining lymph nodes refers to lymph notes that lie immediately downstream of tumors.

Pharmaceutical compositions may be formulated for any appropriate route of administration. For example, compositions may be formulated for intravenous, parenteral, subcutaneous, intramuscular, intracranial, intraorbital, ophthalmic, intraventricular, intracapsular, intraspinal, intracisteral, intraperitoneal, intranasal, or aerosol administration. In some embodiments, pharmaceutical compositions are formulated for direct delivery to the tumor (intratumoral) or to the tumor environment. In another embodiment, pharmaceutical compositions are formulated for delivery to the lymph nodes.

### Compositions

Provided and useful herein are compositions alter neutrophil migration to reduce or inhibit tumor cell seeding, tumor metastasis, or metastatic niche formation. Such compositions are useful in the methods described herein. Useful compositions include those which inhibit the purinergic receptors P2Y₁ and/or P2Y₂. Such compounds are, in some embodiments, known in the art and are alternatively known as antagonists. Such compounds include all inhibitors/antagonists of P2Y1 and/or P2Y2 including, without limitation, peptides, nucleic acid molecules, small molecule compounds, antibodies and derivatives thereof Also provided herein are pharmaceutical compositions that include purinergic receptor antagonists. Without limitation, compositions provided herein include one or more inhibitors identified in the following table:

| CXCR 1 or 2 or | | |
|---|---|---|
| 1/2 Inhibitors- | Pannexin-1 Inhibitors | Connexin Inhibitors |
| Ladarixin | Spironolactone | 18α-glycyrrhetinic acid |
| DF-2755A | Probenecid | 18β- glycyrrhetinic acid |
| Repertaxin (Reparixin) | Tenofovir | Carbenoxolone |
| Reparixin L-Lysine Salt | Brilliant Blue | Heptanol |
| Navarixin | FCF | Octanol |
| DF2162 | 18α- | Halothane |
| SB-225002 | glycyrrhetinic | Oleic Acid |
| SB-332235 | acid | Linoleic Acid |
| SCH-479833 | 18β- | Arachidonic Acid |
| SCH-527123 | glycyrrhetinic | Flufenamic Acid |
| | acid | Quinine |
| | Carbenoxolone | Mefloquine |
| | Arachidonic | 2-APB |
| | Acid | Polyamines |
| | Palmitic Acid | Cyclodextrins |
| | Trovafloxacin | |
| | | P2X1 and P2Y1 and 2 |
| Glut1 Inhibitors | Glycolytic inhibitors | Inhibitors |
| Fasentin | WP-1122 | Suramin hexasodium |
| WZB 117 | Phloretin | NF449 |
| BAY 876 | Quercetin | NF279 |
| STF 31 | STF-31 | GLS-409 |
| 1H-pyrazolo[3,4- | EZB-117 | MR52179 |
| d]pyrimidines | Bromopyruvate | AR-C118925 |
| EF24 | 3PO | BMS-884775 |
| PUG-1 | Dichloroacetate | |
| Naringenin | Oxamic Acid | |
| Myricetin | NHI-1 | |
| Fisetin | | |
| Quercetin | | |
| Isoquercitrin | | |
| Apigenin | | |
| Luteolin | | |
| Kaempferol | | |
| Genistein | | |
| Silybin | | |
| Xanthohumol | | |
| Phloretin | | |
| Phlorizin | | |
| Daidzein | | |

MRS2179 (3′-adenylic acid, 2′-deoxy-N-methyl-5′-(dihydrogen phosphate), ammonium salt) is a competitive P2Y1 receptor antagonist, which inhibits ADP-induced platelet shape change and aggregation (pA2 =6.55) in vitro and prolongs bleeding time in rats and mice compared to controls. MRS2179 has a molecular weight of 452.2 and is available commercially. See Dunne, et al. BMC Proc. 2015; 9(Suppl 1): A2 “MRS2179: a novel inhibitor of platelet function”, which is incorporated herein by reference.

AR-C118925 (5-[[5-(2,8-Dimethyl-5H-dibenzo[a,d]cyclohepten-5-yl)-3,4-dihydro-2-oxo-4-thioxo-1(2H)-pyrimidinyl]methyl]-N-2H-tetrazol-5-yl-2-furancarboxamide) is a selective, competitive P2Y2 receptor antagonist which inhibits P2Y2 receptor-induced β-arrestin translocation in vitro (pA2=37.2-51.3 nM). AR-C118925XX has a molecular weight of 537.59 and is available commercially. See, Rafehi et al., Purinergic Signal. 2017 March; 13(1):89-103 “Synthesis, characterization, and in vitro evaluation of the selective P2Y2 receptor antagonist AR-C118925”, which is incorporated herein by reference.

NF449 (4,4′,4″,4″′-[Carbonylbis(imino-5,1,3-benzenetriyl-bis(carbonylimino))]tetrakis-1,3-benzen edisulfonic acid octasodium salt) is a selective Gsα-subunit G-protein antagonist which reduces the association rate of guanosine 5′-[γ-thio]triphosphate ([35S]GTP[γS]) binding to Gγ s -s (Gs&alpha-s) and inhibits the stimulation of adenylyl cyclase activity in S49 cyc-membranes. In addition, NF 449 interferes with binding between the Adenosine A1-R (A1-adenosine receptor) and its cognate G proteins (Gi/Go). Alternate studies suggest that NF449 inhibits a wide array of P2 receptors such as P2X3, P2Y1, and P2Y2, while showing the highest selectivity towards P2X1. Furthermore, NF449 can disrupt the coupling of β-AR (β-adrenergic receptors). NF449 has a molecular weight of 1505.89 and is available commercially. Adenosine 2′,5′-diphosphate sodium salt is an inhibitor of P2X1 and P2Y1. It has a molecular weight of 427.20 and is available commercially.

Pyridoxalphosphate-6-azophenyl-2′,4′- disulfonic acid (PPADS) tetrasodium salt is a selective P2 purinoceptor antagonist that blocks pre and post junctional responses. PPADS has a molecular weight of 599.3 and is available commercially.

Other purinergic receptor antagonists are described in publications such as US Patent Publication No. 20180271863, which is incorporated herein by reference. In some embodiments, the purinergic receptor antagonist includes AF-353, A317491, AF-219, suramin, MRS2159, AR-C118925, GLS-409, PSB-1011, NF770, A740003, RB2, MRS2279, MRS2500, MRS2578, NF340, PSB0739, or PPTN.

In other embodiments, the purinergic compound is ATP, ADP, or a combination of ATP and ADP. Examples of pharmaceutical compositions, according to the invention, include reactive red 2, 8,8′-(carbonylbis(imino-3,1-phenylene carbonylimino)bis(1,3,5-naphthalenetrisulfonic acid), 8,8′-(carbonylbis(imino-4,1-phenylenecarbonylimino-4,1-phenylene-carbonylimino)) bis(1,3,5-naphthalenetrisulfonic acid), Evans blue, trypan blue, reactive blue 2, pyridoxalphosphate-6-azophenyl-29,49-disulfonic acid, isoquinoline sulfonamide, 1-[N,O-bis(5-isoquinoline-sulfonyl)-N-methyl-L-tyrosyl]-4-phenylpiperazine, trinitrophenyl-substituted nucleotides, diinosine pentaphosphate, cicacron blue 3GA, 2′,3′-O-(2,4,6-trinitrophenyl)-ATP, substance P, basilen blue, and 4,4′-diisothiocyanostilbene-2,2′-disulfonic acid, or pharmaceutically acceptable salts thereof. See US US20040029841, which is incorporated herein by reference.

Other compositions useful herein include inhibitors of pannexin-1 (also called Panx1) or connexin hemichannels. Such compounds are, in some embodiments, known in the art and are alternatively known as antagonists. Also provided herein are pharmaceutical compositions that include pannexin-1 or connexin antagonists.

Pannexin inhibitors include those described in US Patent Application No. 2018/0028595, which is incorporated herein by reference, including, without limitation, probenecid, carbenoxolone, 10Panx1, mefloquine, 5-nitro-2-(3-phenylpropylamino)benzoic acid, and trovafloxacin. In another embodiment, the pannexin inhibitor is a peptide or small molecule that targets the YLK sequence of pannexin 1, such as the IL2 peptide described in US Patent Application No. 2018/0028595. Other pannexin-1 inhibitors are described in WO 2013/111014, which is incorporated herein by reference. Such inhibitors include pannexin-1 siRNA or anti-pannexin-1 antibodies or fragments thereof. Other inhibitors include pannexin-1 peptides, such as IOPanx, P308 peptide, or a pannexin-1 binding partner peptide. In another embodiment, the pannexin-1 inhibitor is tenofovir. In another embodiment, the pannexin-1 inhibitor is spironolactone.

Other compositions useful herein include ER stress antagonists. Such compounds are, in some embodiments, known in the art and are alternatively known as inhibitors. Also provided herein are pharmaceutical compositions that include ER stress antagonists.

ER stress antagonists include, without limitation, small molecule modulators of BI09, including CDN-1163, SERCA2b, TDI-194, AC-915, RX-1, ADoPep-2, and AC-2010 all of which are known in the art. Other ER stress inhibitors include those described in US20180059115, which is incorporated herein by reference. Others include B-I09, and its derivatives, which targets the IRE-1/XBP-1 pathway. See, Tang et al., J Clin Invest. 2014 Jun;124(6):2585-98. doi: 10.1172/JCI73448 “Inhibition of ER stress-associated IRE-1/XBP-1 pathway reduces leukemic cell survival” which is incorporated herein by reference.

Pharmaceutical compositions may be in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc. Methods and agents well known in the art for making formulations are described, for example, in “Remington's Pharmaceutical Sciences,” Mack Publishing Company, Easton, Pa. Formulations may, for example, contain excipients, diluents such as sterile water, or saline, polyalkylene glycols such as polyethylene glycol, oils of vegetable origin, or hydrogenated napthalenes.

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### EXAMPLE 1

Here, we describe the two-phase pathological activation of neutrophils in the bone marrow of tumor-bearing mice and in the blood of cancer patients. The first phase is characterized by an accumulation of MDSC-like neutrophils that lacked immunosuppressive activity but displayed profound up-regulation of glucose metabolism, ATP production and a potent ability to spontaneously migrate. The second phase is characterized by the accumulation of neutrophils with typical features of PMN-MDSCs that, however, were indistinguishable from control neutrophils with regards to their metabolic activity and migratory behavior.

### Methods

### Human Subjects and Samples

Samples of peripheral blood were collected from patients at the Helen F. Graham Cancer Center. The study was approved by the Institutional Review Board (IRB) of the Christiana Care Health System at the Helen F. Graham Cancer Center and The Wistar Institute. All patients signed IRB approved consent forms. Samples were collected at Helen F. Graham Cancer Center from 18 patients with previously untreated stage II-IV non-small cell lung cancer (NSCLC) and 8 patients with stage III-IV head and neck cancer. This cohort includes 14 females and 12 males, aged 48-74 years.

### Mice Models and Mice Treatments

Female and male C57BL/6N CD45.1⁺ and female C57BL/6 CD45.2⁺ mice (aged 6-8 weeks) were purchased from Charles River Laboratories. Female OT-I TCR-transgenic mice (C57B1/6-Tg(TCRaTCRb)1100mjb) (4-6 week old) and female Pmel TCR-transgenic mice (B6.Cg-Thy1^{a}/Cy Tg(TcraTcrb)8Rest) (4-6 week old) were purchased from Jackson Laboratories. Female C57B1/6 CD45.1⁺/2⁺ were generated by crossing a male CD45.1⁺ with a female CD45.2⁺. All the mice were housed in pathogen-free conditions and handled in accordance with the requirements of the guidelines for animal experiments. The research was approved by the Wistar Institutional Animal Care and Use Committee. For in vivo migration experiments, 1×10⁶ CD45.1⁺ Ly6G⁺ BM cells were mixed with 1×10⁶ CD45.2⁺ Ly6G⁺ BM cells in a total volume of 100 μL of PBS and injected intravenously in female C57B1/6 CD45.1⁺/2⁺ mice.

### Cell Lines

EL4 lymphoma, LLC (Lewis Lung Carcinoma), CT26 colon carcinoma were purchased from ATCC. The LL2 (Lewis Lung Carcinoma) tumor cell line expressing luciferase was a gift from Rupal Ramakrishnan (H. Lee Moffitt Cancer Center). They were maintained in DMEM medium supplemented with 10% fetal bovine serum (FBS, Sigma-Aldrich) and 1% penicillin-streptomycin (ThermoFisher Scientific) at 37° C. with 5% CO₂. Tumors cells were injected intravenously at 5×10⁴ cells per mouse in a total volume of 100 μL of PBS.

### Mouse Neutrophil and PMN-MDSC Isolation

Legs bones (tibias and femurs) cleaned of muscular tissues are cut at both ends. Bone marrow (BM) cells are then flushed out with a 25-gauge needle and a 5 mL syringe filled with a cold solution of PBS 1× (ThermoFisher Scientific), FBS 1%, EDTA 2 mM (ThermoFisher Scientific) (cell suspension buffer, CSB). Cell suspension is then filtered through a 70 μm strainer (Fischer Scientific) placed on a conical 50 mL Falcon tube. For spleens, the organ is put in a 70 μm strainer placed on a conical 50 mL Falcon tube and cut into small pieces. These pieces are then grinded against the cell strainer using the plunger of a 5 mL syringe and washed several times with cold CSB. Tubes are then centrifugated at 1500 rpm at 4° C., the supernatant is removed and red blood cells are lysed by resuspending the cell pellet in ammonium chloride lysis buffer for 5 minutes at room temperature. Cells are then washed with cold CSB, spin down and the pellet is resuspended in cold CSB and counted using Trypan blue (VWR). Single-cell suspensions from lungs were prepared using mouse lung dissociation kit (Miltenyi Biotec) according to the manufacturer's recommendations with an additional red blood cell lysis step as described above. For BM Ly6G⁺ cells isolation, cells were labeled with biotinylated anti-Ly6G antibody (Miltenyi Biotec), incubated with streptavidin-coated microbeads (Miltenyi Biotec) and separated on MACS columns (Miltenyi Biotec).

### Transwell Assays for Migration and Chemotaxis

Unstimulated migration and chemotaxis was measured using a 3 μm pore transwell system (Neuro Probe Inc. or Sigma-Aldrich). Advanced RPMI with CXCL1 (BioLegend) or fMLP (Sigma-Aldrich) was placed in the bottom of the transwell, as indicated. 0.1×10⁶ or 0.5×10⁶ cells were incubated with pannexin inhibitor (¹⁰Panx; Tocris), scrambled ¹⁰Panx (Tocris), pan-P2XR inhibitor suramin (R&D Systems), P2X1 inhibitor NF 449 (Tocris), A3R inhibitor MRS 1191 (Santa Cruz), or apyrase (New England Biolabs) in Advanced RPMI for 10 min prior to being plated on top of the Neuro Probe system or Sigma-Aldrich system filter, respectively, and placed at 37° C., 5% CO₂ for 1 hour. 10 μl media was taken from the bottom wells and counted by hemocytometer. The quantification of migrated neutrophils was done using the formula N=n×10⁴×0.029 reflecting 29 μ1 media in the bottom well.

### Seahorse Assay

Metabolic rates were determined using the Seahorse XF24 and XF96 Flux Analyzers (Seahorse Biosciences) following the manufacturer's protocol. Briefly, the microplate was coated with 22.4 μg/ml Cell-Tak (Fisher) using 200 mM sodium bicarbonate. 1.2×10⁶ or 0.4×10⁶ cells were seeded per well immediately after isolation in 50 μl and 80 μl of unbuffered RPMI (Sigma-Aldrich) for the XF24 and XF96 analyzers, respectively. The microplate was incubated for 30 min at 37° C. to allow the cells to settle into a monolayer. Unbuffered RPMI was gently added to the wells without disturbing the monolayer to bring the assay volume to 675 μl and 180 μl for the XF24 and XF96 analyzer, respectively. The basal oxygen consumption rate (OCR) and extracellular acidification rate (ECAR) was measured, in addition to rate changes upon treatment with 5 μM oligomycin (Sigma-Aldrich), 1 μM FCCP (Sigma-Aldrich), and 0.75 μM rotenone and 1 μM antimycin A (Sigma-Aldrich).

Metabolomics

Neutrophils, PM-LC and PMN-MDSC cells were isolated from bone marrow and resuspended in our homemade culture medium which contains nutrients at physiological concentrations, and was previously described⁴⁵. Cells were allowed to adjust to the new medium for 1 hr before 5.5 mM of ¹³C₆-glucose was spiked into the medium. Cells were incubated for a further 90 min with the stable isotope tracer and then harvested. For harvesting, cell pellets were washed twice in ice-cold PBS and extracted in a solution of LC-MS grade methanol, acetonitrile, and ultrapure water. Samples were centrifuged and the resulting cleared supernatant was transferred to a silanized MS vial and run by LC-MS. For analysis of nutrient uptake and efflux, the medium was removed and diluted 50-fold into extraction solution and vortexed on a thermomixer for 10 min at 4° C. before being frozen at −80° C. overnight. The extracted medium was thawed on ice the next day and following centrifugation, the cleared supernatants were transferred to silanized glass vials and either run immediately by LC-MS or stored at −80° C. Metabolite measurements were normalized based upon protein concentration determined from cell pellets. LC-MS metabolite flux analysis was performed on a Thermo Scientific Q Exactive Plus mass spectrometer equipped with a HESI II probe and coupled to a Shimadzu Nexera UHPLC system. Column and LC conditions are as described above. The mass spectrometer was operated in full-scan, polarity switching mode with the spray voltage set to 3.2 kV in positive ion mode and 2.5 kV in negative ion mode. The heated capillary was set at 275° C., the HESI probe at 350° C., and the S-lens RF level at 45. The gas settings for sheath, auxiliary and sweep were 40, 10 and 1 unit, respectively. The mass spectrometer was set to repetitively scan m/z from 70 to 1000, with the resolution set at 70,000, the AGC target at 1E6, and the maximum injection time at 80 ms. Metabolite identification and quantitation was performed with TraceFinder 3.1 software (Thermo Fisher Scientific).

### Real-Time Quantitative PCR

Cells were lysed and RNA was isolated using the E.Z.N.A. total RNA purification kit (Omega Bio-Tek). Reverse transcription was performed using the High Capacity cDNA Reverse Transcription Kit (Applied Biosystems Inc.). Quantitative PCR was then performed using Sybr Green PCR Master Mix (Applied Biosystems Inc.) on an ABI 7500 Fast instrument.

Glut1
Fwd:
(SEQ ID NO: 1)
AGCCCTGCTACAGTGTAT
Rev:
(SEQ ID NO: 2)
AGGTCTCGGGTCACATC
Glut3
Fwd:
(SEQ ID NO: 3)
ATGGGGACAACGAAGGTGAC
Rev:
(SEQ ID NO: 4)
CAGGTGCATTGATGACTCCAG
HIF1α
Fwd:
(SEQ ID NO: 5)
TCTCGGCGAAGCAAAGAGTC
Rev:
(SEQ ID NO: 6)
AGCCATCTAGGGCTTTCAGATAA
Hexokinase
Fwd:
(SEQ ID NO: 7)
ATGATCGCCTGCTTATTCACG
Rev:
(SEQ ID NO: 8)
CGCCTAGAAATCTCCAGAAGGG
Phosphofructokinase
Fwd:
(SEQ ID NO: 9)
CATCGCCGTGTTGACCTCT
Rev:
(SEQ ID NO: 10)
CCCGTGAAGATACCAACTCGG
GAPDH
Fwd:
(SEQ ID NO: 11)
CCCTTAAGAGGGATGCTGCC
Rev:
(SEQ ID NO: 12)
ACTGTGCCGTTGAATTTGCC
Phosphoglycerate kinase
Fwd:
(SEQ ID NO: 13)
CCCAGAAGTCGAGAATGCCTG
Rev:
(SEQ ID NO: 14)
CTCGGTGTGCAGTCCCAAA
Enolase
Fwd:
(SEQ ID NO: 15)
AGTACGGGAAGGACGCCACCA
Rev:
(SEQ ID NO: 16)
GCGGCCACATCCATGCCGAT
Pyruvate kinase
Fwd:
(SEQ ID NO: 17)
GCCGCCTGGACATTGACTC
Rev:
(SEQ ID NO: 18)
CCATGAGAGAAATTCAGCCGAG
β-actin
Fwd:
(SEQ ID NO: 19)
CCTTCTTGGGTATGGAATCCTGT
Rev:
(SEQ ID NO: 20)
GGCATAGAGGTCTTTACGGATGT

### F-Actin Measurement

Total bone marrow cells were stained with AQUA, CD11b-BV421, and Ly6G-APC. Cells were washed and stimulated with doses of CXCL1 and fMLP in Advanced RPMI for the time points indicated. Cells were immediately fixed and permeabilized using Cytofix/Cytoperm Solution (BD) and washed in Perm/Wash Buffer (BD) following the manufacturer's protocol. Cells were stained with 1 unit of Phalloidin-AF488 (Thermo Fisher) for 20 min at 4° C., washed in Perm/Wash Buffer, run on a flow cytometer, and analyzed using FlowJo.

### Isolation of Human Neutrophils and PMN-MDSCs

For isolation of total population of human neutrophils from healthy individuals and cancer subjects we used MACSxpress isolation kit (Miltenyi). For parallel isolation of PMN-MDSC and neutrophils, double density gradient of Histopaque-1077 and Histopaque-1119 (Sigma Aldrich) was used. PMN-MDSC were isolated from low density PBMC using CD15-beads (Miltenyi). Neutrophils were isolated from high density gradient also using CD15-beads. Neutrophil and PMN-MDSC purity was assessed by flow cytometry and was >95%.

### Flow Cytometry

All antibody incubations were performed for 15 minutes at 4° C. in dark and centrifugations done at 1500 rpm at 4° C. for 5 minutes, unless recommended otherwise by the manufacturer. Usually up to 1×10⁶ cells were incubated with Fc-block (BD Biosciences; clone 2.4 G2 ; cat. no. 553142) in 50 μL of CBS, then washed in CBS and spin down before cell surface staining with additional antibodies. After the last incubation, cells were washed in CBS, spin down and resuspended in 400 μL of CBS before acquisition. Cells were run on LSRII flow cytometer (BD Biosciences) and data were analyzed by FlowJo (Tristar). For in vivo migration experiments analyses, the whole organ (spleen or lungs) was stained in the same way as described above except that up to 1×10⁷ cells were stained in 50 μL of CBS. Flow cytometry reporting summary can be found in Life Sciences Reporting Summary.

### RNA-Seq

Neutrophils were isolated using Ly6G beads with purity was >95%. RNA sequencing was performed using Illumina Hiseq 2500 platform (Illumina, San Diego, Calif., USA). VAHTS Total RNA-Seq Library Preparation Kit was used for library preparation. Single-end read runs were used, with read lengths up to 50 bp in high output mode, 30M total read counts. Data was aligned using RSEM v1.2.12 software⁴⁶ against mm10 genome and gene-level read counts and RPKM values on gene level were estimated for ensemble transcriptome. Samples with at least 80% aligned reads were analyzed. DESeq2⁴⁷ was used to estimate significance between any two experimental groups. Overall changes were considered significant if passed FDR<5% thresholds with an additional threshold on fold change (fold>5) taken to generate the final gene set. Gene set enrichment analysis was done using QIAGEN's Ingenuity® Pathway Analysis software (IPA®, QIAGEN Redwood City, www.qiagen.com/ingenuity) based on “Functions”, “Canonical Pathways”, “Upstream Regulators” and “Networks” options. RNA-seq data were deposited to GEO data repository, accession number GSE118366.

### Suppression Assays

After BM Ly6G+ cells isolation as described above, cells were plated in U-bottom 96-well plates in triplicates in complete RMPI without extra cytokines. They were co-cultured at different ratios with total splenocytes from Pmel or OT-1 transgenic mice in the presence of cognate peptides: OT-1, SIINFEKL; Pmel, EGSRNQDWL. Cells were incubated for 48 hours and then ³H thymidine (PerkinElmer) was added (1 μl/well) and incubated overnight. Samples were counted with a TopCount NXT instrument (PerkinElmer).

### In Vivo Tumor Experiment

In the short-term lung metastasis model, LLC were labelled with CFSE (BioLegend). 1×10⁶ labelled LLC cells were intravenously injected 5 hours after i.v injection of 1×10⁶BM Ly6G⁺ cells isolated from naive mice, 1 week, or 3 weeks LL2 TB mice. At the time indicated, mice were sacrificed and lungs were collected after perfusion with PBS to eliminate circulating tumor cells. Lungs were cut into small pieces and digested using Lung Dissociation Kit according to the manufacture's protocol (Miltenyi Biotec). Subsequently, the lungs were passed through a 70um cell strainer and subjected to red blood cell lysis using ammonium-chloride-potassium (ACK) buffer. Lung single cell suspensions were stained with CD45-APC-Cy7 (BD Biosciences). For long-term lung metastasis model, 5×10⁴ LL2 cells were intravenously injected 5 hours after i.v injection of 2×10⁶BM Ly6G⁺ cells isolated from naive mice, 1-week, or 3-weeks LL2 TB mice. Mice were sacrificed 14 days after injection and the tumor burden was analyzed. The luciferase signal was measured in excised lungs with an IVIS Spectrum imaging system (Caliper).

### Time-Lapse Migration Assay

The wells of a 12-well plate were coated with 50 μg/ml fibronectin (Sigma-Aldrich, St. Louis, Mo.) and washed with PBS. 0.5×10⁶ cells were plated per well in Advanced RPMI (Thermo Fisher, Cat #:12633-012) without FBS and placed in a 37° C., 5% CO₂ incubator for 10-15 minutes to allow for cell attachment. The wells were gently washed with PBS 2X to remove non-adherent cells and Advanced RPMI was added to the wells. Cells were placed onto a motorized stage and observed using a Nikon Eclipse TE300 fluorescence microscope maintained in an environment of 37° C. and 5% CO₂. A 10× or 20× objective was used to capture images during the course of the time lapse. Images were captured every 30 seconds over the course of 15 minutes from at least four different fields of view.

### Measurement of Cell Trajectories and Mean-Squared Displacements

Cell movement was tracked using the ImageJ plugin Manual Tracking. ImageJ and the plugin are both freely available through the NIH website (http://rsbweb.nih.gov/ij/). The centroid of the cell was considered to represent the cell position. Time lapse microscopy was used, and images were taken every 1.5 minutes. The result was a series of (x,y) positions with time for each cell. The net displacement during the ith 1.5-minute increment, Dᵢ, was calculated by the difference of the position at the beginning and end of that time step. The mean-squared displacement, [symbol] D²(t) [symbol] , over time was calculated using the method of non-overlapping intervals. Speed, S, can be considered as the total path length over time and persistence time, P, is the time a cell remains moving without changing direction. S and P were obtained by fitting these to the persistent random walk equation [symbol] D²(t) [symbol] =2S²[t−P(1−e^{-t/P})] where t is the time interval, using a non-linear least squares regression analysis ^{48, 49}. The random motility coefficient (μ) was then calculated as μ=½S²P⁵⁰

### Cytokine Protein Array

Expression of cytokines in mouse sera was evaluated using mouse cytokine antibody array Panel A (ARY006, R&D Systems). Sera from one-week and three-week LL2 TB mice were mixed with a cocktail of biotinylated detection antibodies followed by streptavidin-labeled horseradish peroxidase and then visualized using chemiluminescence-based detection. Densities of the spots were quantified with Image J.

### Statistics

Statistical analyses were done using two-tailed unpaired Student's t tests. In mean-squared displacement two-way ANOVA with Bonferroni adjustment for multiple comparisons was used. Statistical tests were performed using GraphPad Prism version 7.0. P values of 0.05 were considered significant. RNAseq data were analyzed by RSEM v1.2.12 software, DESeq2, Ingenuity® Pathway Analysis software.

### Data Availability

The data that support the findings of this study are available from the corresponding author upon request. Source data for each figure are provided in supplement. RNAseq data are deposited to GEO data repository, accession number GSE118366.

### EXAMPLE 2

### Results

### Enhanced Motility of BM Neutrophils in Tumor-Bearing Mice Depends on Stage of Tumor

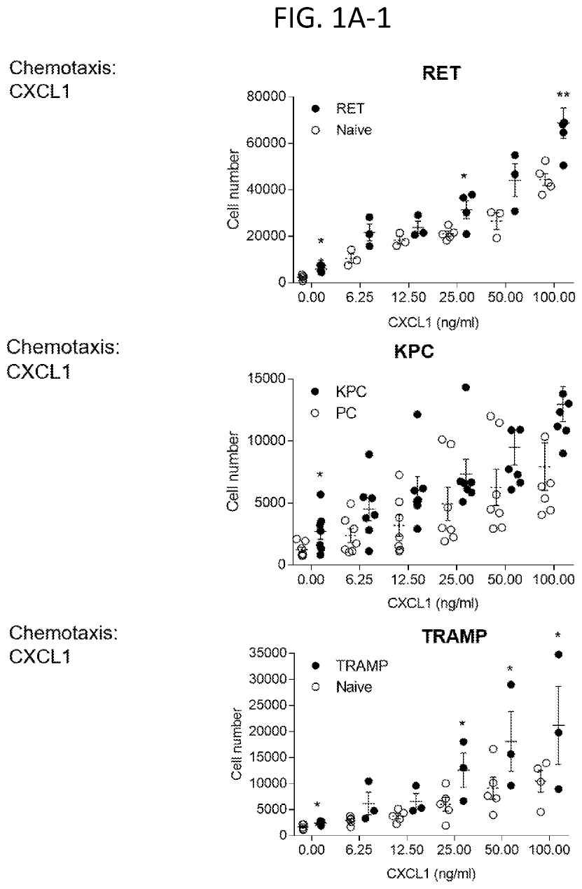

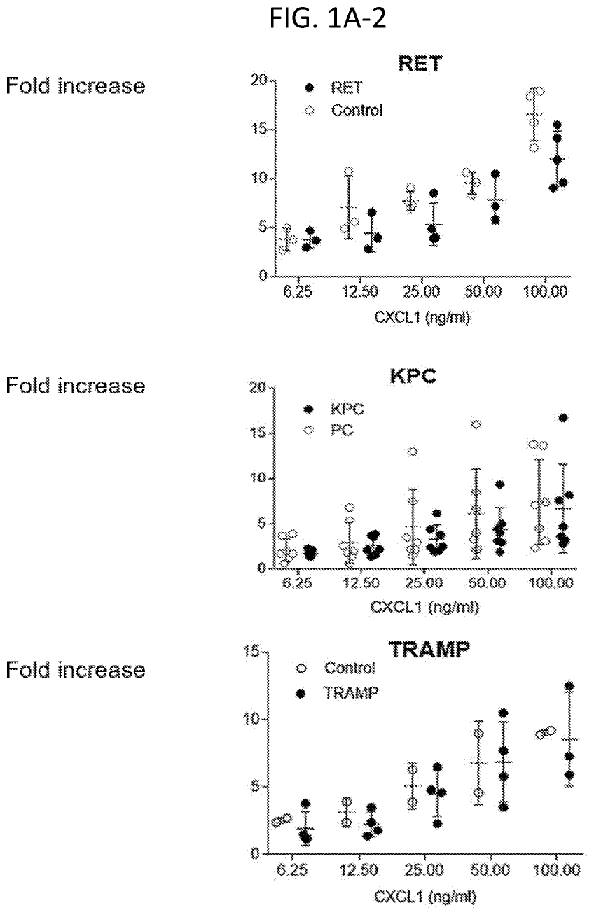

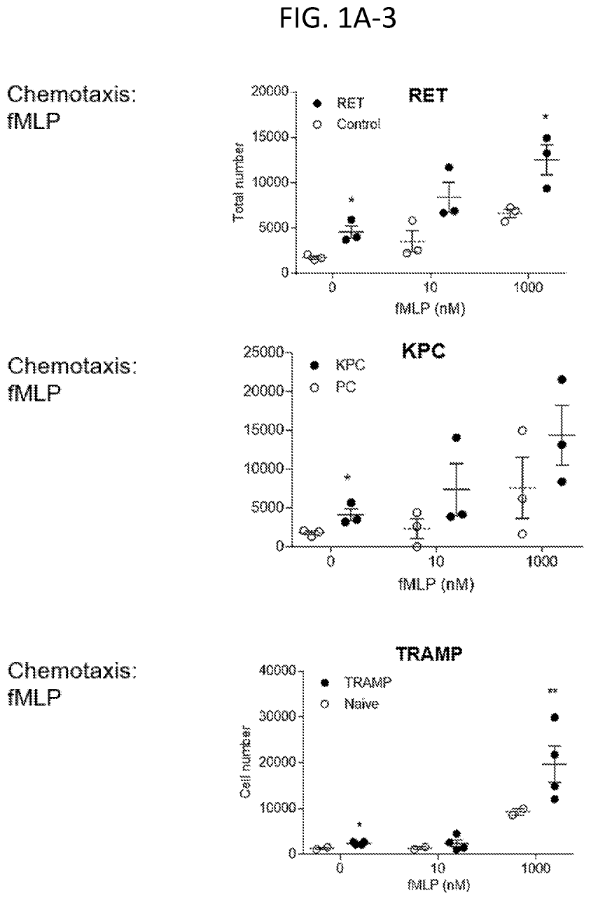

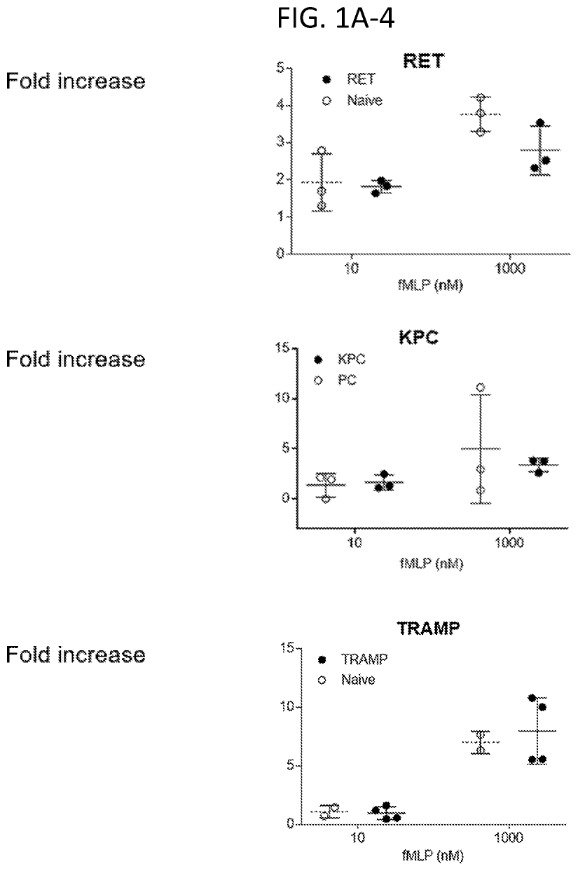

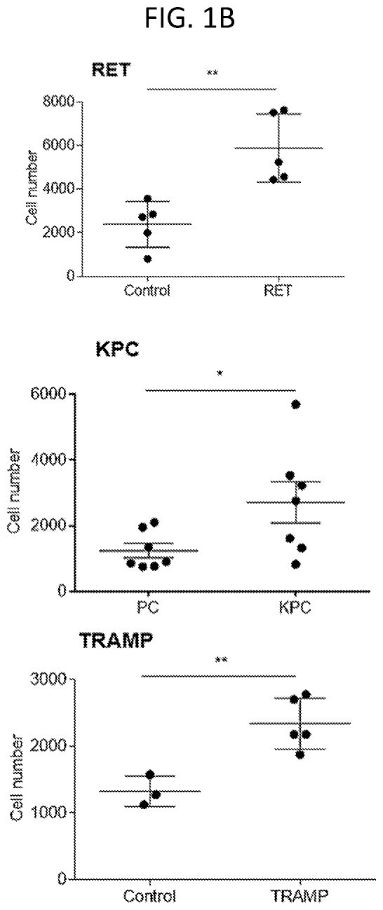

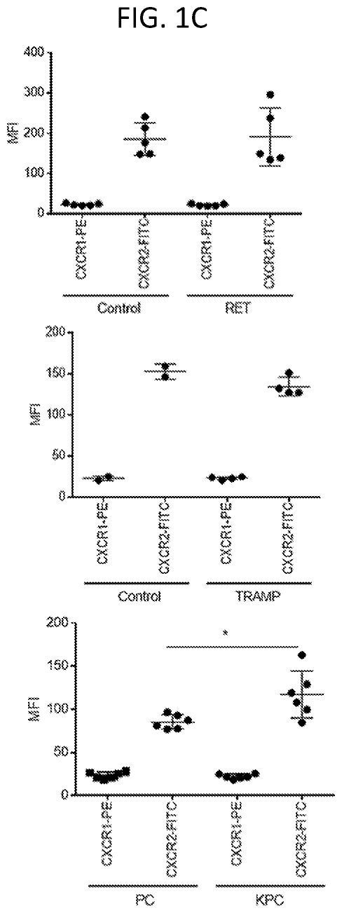

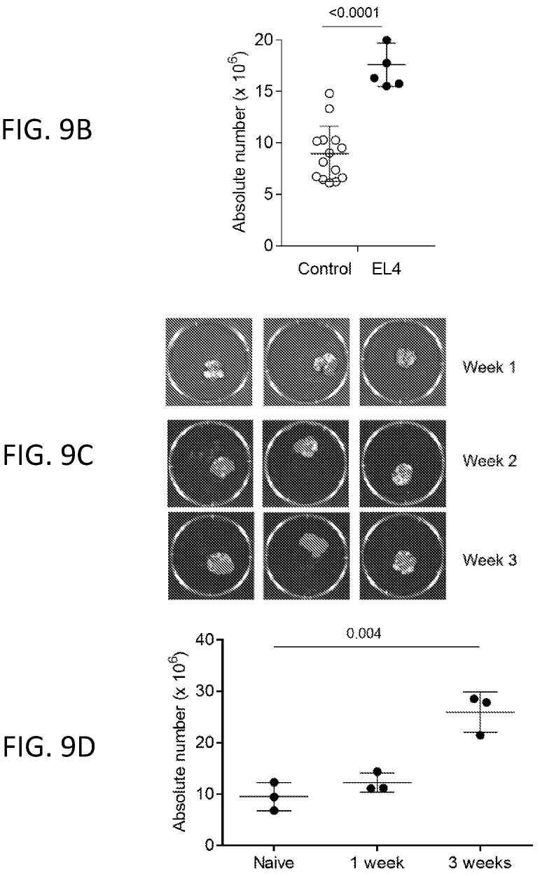

We evaluated the migration of neutrophils isolated from the BM of three different genetically-engineered models (GEM) of cancer: RET melanoma²⁴, KPC pancreatic cancer²⁵, and TRAMP prostate cancer²⁶. These mice were backcrossed for more than 10 generations to the C57BL/6N background. Neutrophils were phenotypically characterized as CD11b⁺Ly6C^{lo}Ly6G⁺ and were isolated based on the expression of the Ly6G marker. Migration and chemotaxis of neutrophils were assessed using a standard Transwell membrane assay. In all three models, we observed substantially higher migration of neutrophils from tumor-bearing mice in response to the chemokine CXCL1 or the chemoattractant fMLP than neutrophils from tumor-free littermates (FIG. 1A). However, when the ability of these stimuli to induce cell migration (ratio between stimulated and unstimulated cells) was evaluated, no differences between TB and tumor-free mice were observed (FIG. 1A). Instead, we observed markedly higher spontaneous migration of neutrophils in tumor-bearing than in tumor-free mice (FIG. 1B). No or very small differences in the expression of the CXCL1 receptors, CXCR1 and CXCR2 were evident (FIG. 1C) consistent with the results of neutrophil chemotaxis. Though the total number of neutrophils was significantly elevated in the BM of KPC pancreatic mice, the total number of neutrophils in RET melanoma and TRAMP prostate mice was similar to control mice (FIG. 9A), suggesting that changes in motility were not associated with expansion of neutrophils in BM.

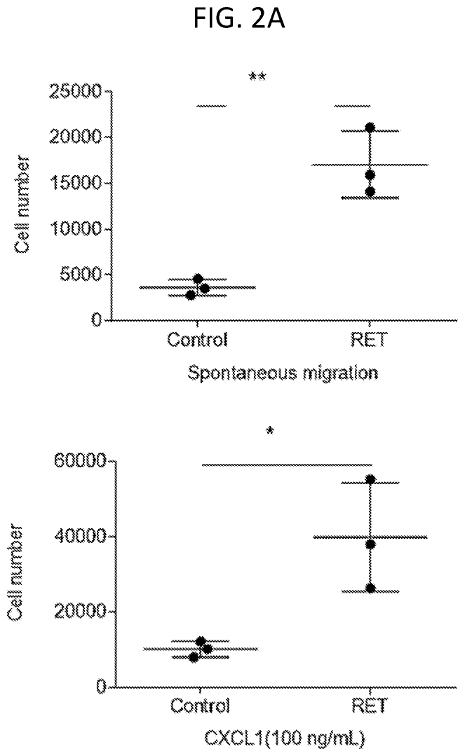

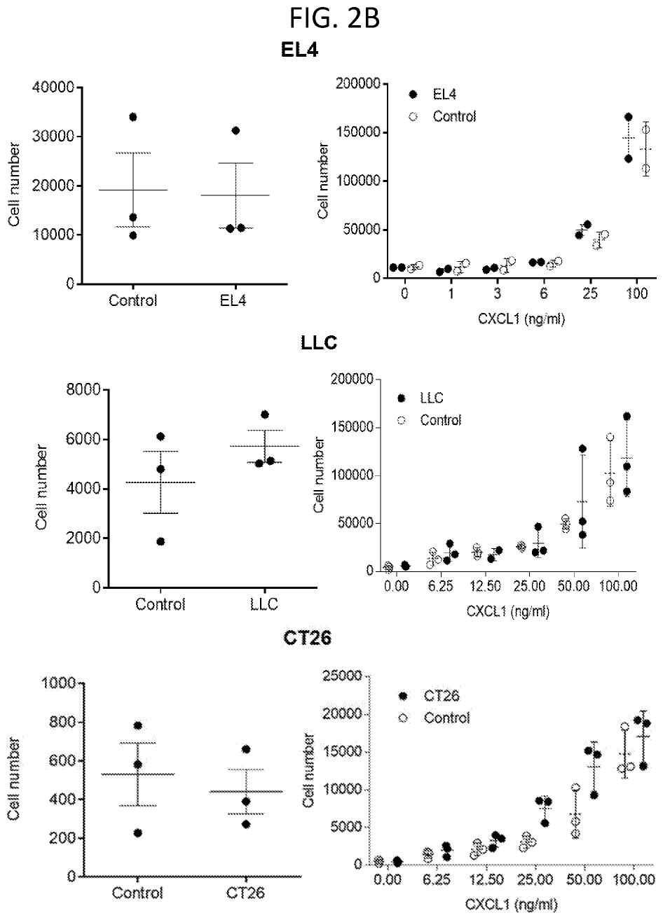

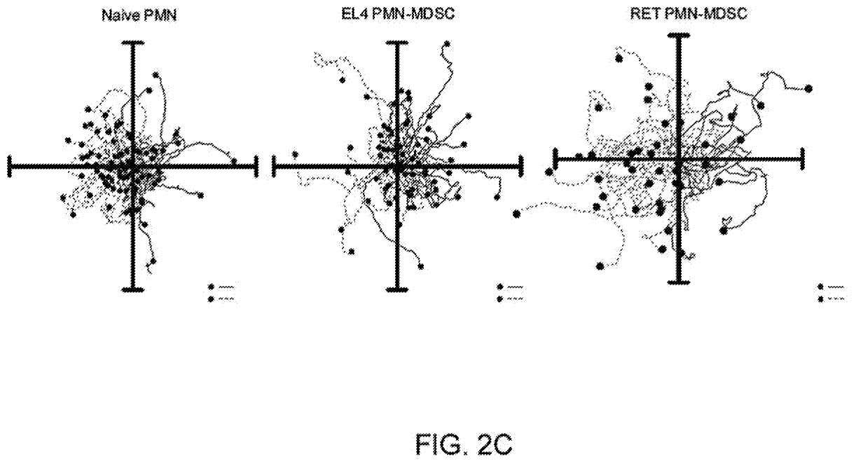

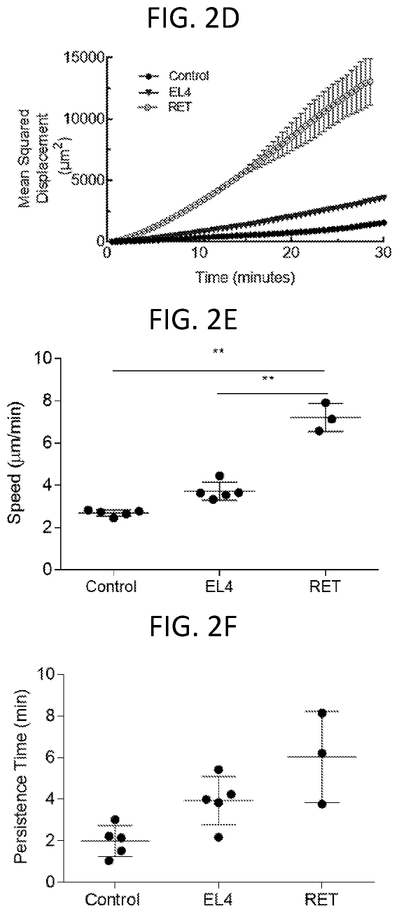

To confirm that differences in migration was not only limited to the BM, we isolated neutrophils from the peripheral blood of control and RET melanoma mice. Neutrophils from RET melanoma mice demonstrated markedly higher spontaneous and CXLC1-induced migration than control neutrophils (FIG. 2A). When we evaluated the migratory behavior of BM neutrophils from transplantable subcutaneous (s.c.) tumor models, EL4 lymphoma, LLC lung carcinoma, and CT26 colon carcinoma, no differences in migration between control and tumor-bearing mice were observed (FIG. 2B). The number of BM neutrophils in EL4 mice was markedly higher than in control mice (FIG. 9B). We analyzed cell movement using time lapse microscopy on fibronectin-coated surfaces. BM neutrophils from RET melanoma mice, but not from EL4 mice, demonstrated a higher capacity to spontaneously migrate than control neutrophils (FIG. 2C), and was characterized by markedly higher mean-squared displacement over time (FIG. 2D), speed (FIG. 2E), persistence time (FIG. 2F) and random motility coefficient (FIG. 2G).

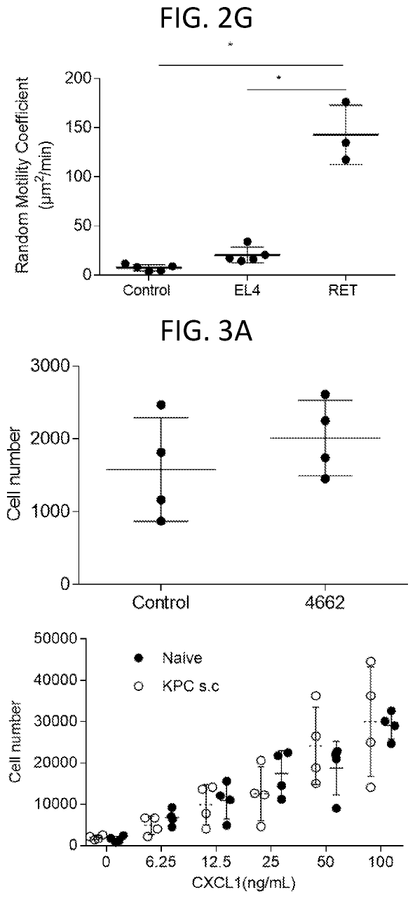

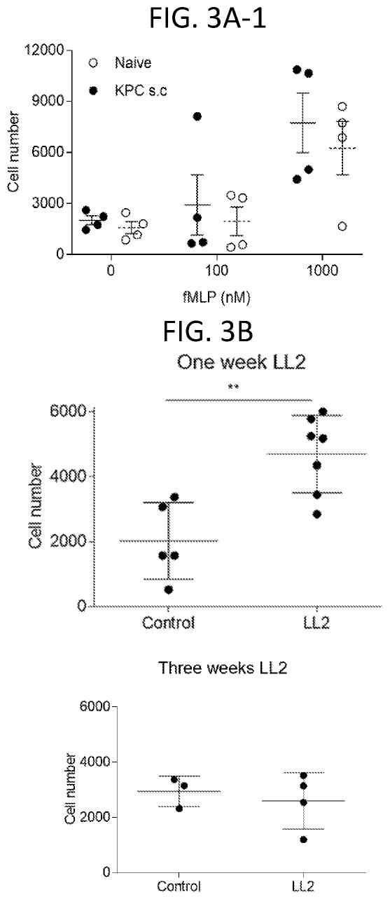

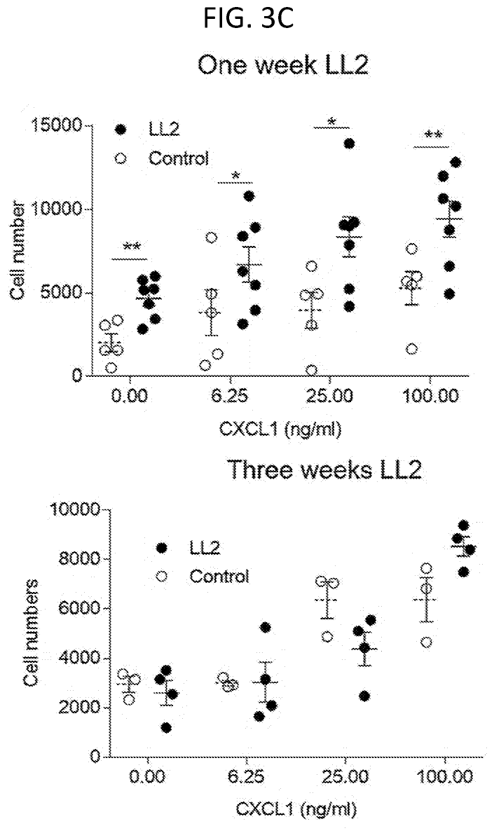

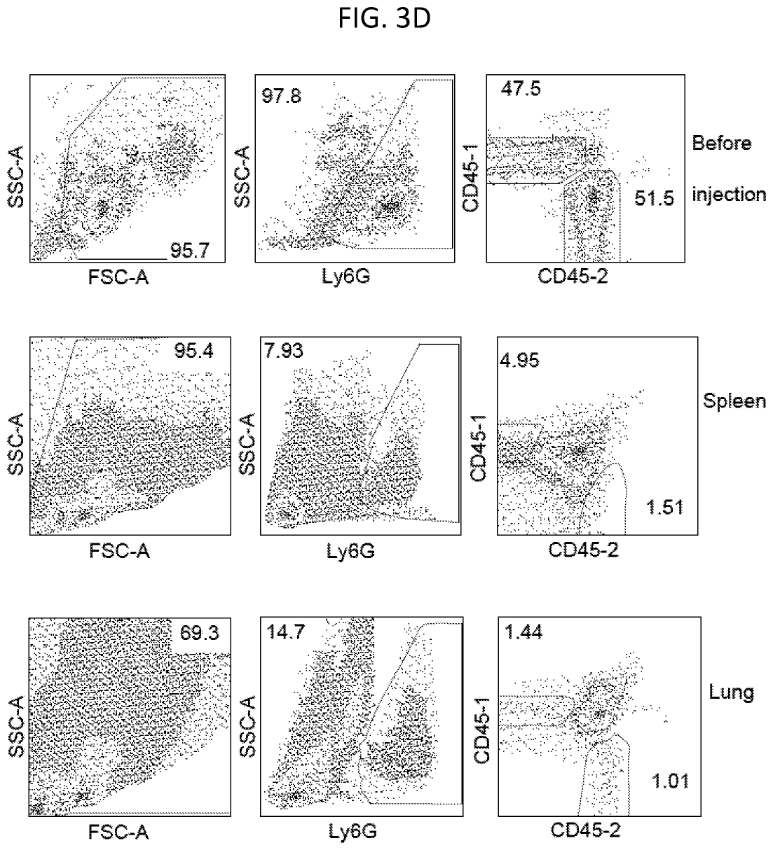

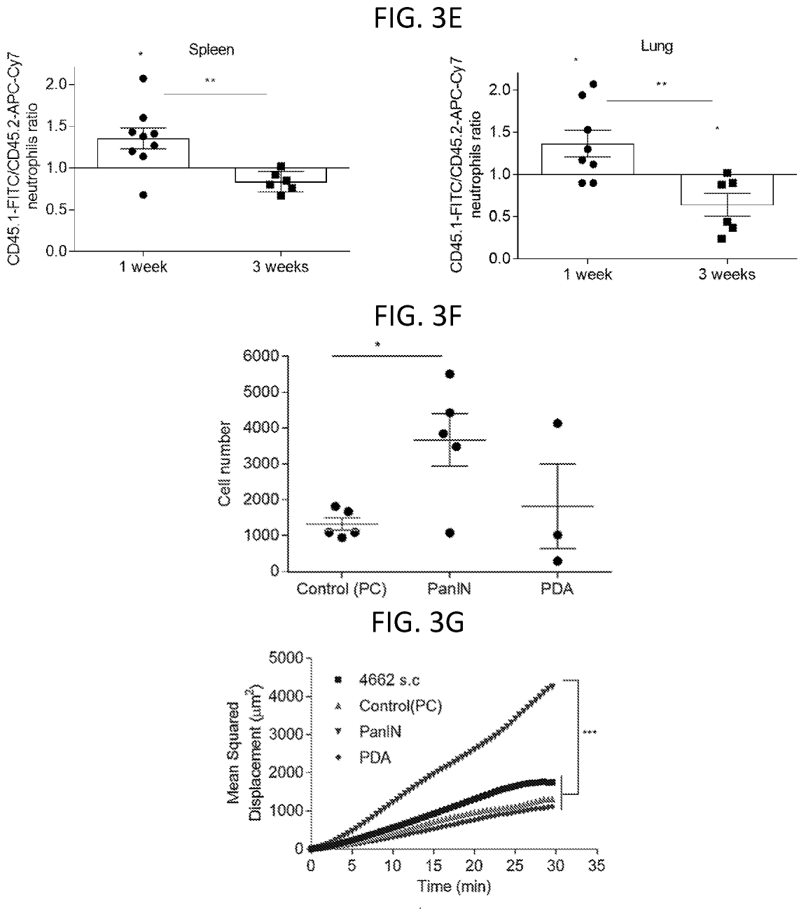

To confirm that how the tumor is established in mice has a critical effect on neutrophil motility, we investigated the migratory behavior of BM neutrophils from a transplantable tumor model established by subcutaneously injecting 4662 a subline of tumor cells derived from KPC mice²⁷. In contrast to neutrophils from KPC mice, neutrophils from 4662 mice did not display increased spontaneous or chemokine-induced migration (FIG. 3A).

We hypothesized that inflammation caused by injecting a large number of tumor cells at the ectopic site could explain the lack of enhanced spontaneous migration of neutrophils in s.c. transplantable models. To test this hypothesis, we utilized an orthotopic model of lung cancer by injecting a small number (10⁵) of LL2 tumor cells containing luciferase (LL2-Luc) intravenously (i.v.), which resulted in the formation of tumor lesions in the lung within one week of injection and large lesions three weeks after injection (FIG. 9C). The total number of BM neutrophils was substantially increased three weeks, but not one week after injection (FIG. 9D). BM neutrophils from mice one week after injection had markedly higher spontaneous and CXCL1-induced migration than control BM neutrophils (FIG. 3B and FIG. 3C). However, when cells were isolated three weeks after injection, no differences were observed (FIG. 3B and FIG. 3C). To verify these observations in vivo, we isolated BM neutrophils from congenic LL2 TB (CD45.1⁺) and control tumor-free (CD45.2⁺) mice, mixed them at a 1:1 ratio, and injected them i.v. into CD45.1⁺×CD45.2⁺ tumor-free recipients. The ratio between CD45.1⁺ and CD45.2⁺ neutrophils was evaluated in spleens and lungs one hour after injection (FIG. 3D). In both tissues, neutrophils from one-week mice were more prevalent than control neutrophils, indicative of increased migratory ability. In contrast, the ratio between neutrophils from three-week and control mice was markedly lower in spleens and lungs (FIG. 3E).

We asked whether association between stage of tumor development and neutrophil activity is observed in mice with GEM of cancer. KPC model could be used to address this question. By 8 to 10 weeks of age, KPC mice develop pancreatic intraepithelial neoplasia (PanIN). KPC mice develop spontaneous pancreatic ductal adenocarcinoma (PDA) with 100% penetrance²⁵, and by 16 weeks of age, most KPC mice have developed locally invasive PDA. Therefore, we were able to separately evaluate mice at the relatively early (PanIN) and relatively late (invasive PDA) stage of pancreatic cancer development. We found that only mice with PanIN, but not invasive PDA, had increased spontaneous migration of neutrophils assessed by Transwell assay (FIG. 3F) and mean squared displacement assay (FIG. 3G).

Thus, in GEM of melanoma, pancreatic, and prostate cancer and at early stages of orthotopic lung cancer, BM neutrophils demonstrated a high capacity to spontaneously migrate. In contrast, BM neutrophils from the s.c. transplantable tumor models and the three-week orthotopic lung cancer model (with substantial tumor burden) exhibited migratory activity similar to control neutrophils.

### BM Neutrophils in the Early and Late Stages of Lung Cancer are Functionally Different

To investigate the differences between mouse BM neutrophils in early and advanced stages of orthotopic lung cancer, we performed gene expression profiling using RNA-seq. Neutrophils from three-week tumor-bearing mice had more significantly changed genes from control mice than neutrophils from one-week tumor-bearing mice (FIG. 4A). Ingenuity Pathway Analysis (IPA) revealed that the most enriched network in BM neutrophils from one-week, but not three-week, LL2 TB mice was Energy Production, Nucleic Acid Metabolism, and Small Molecule Biochemistry (FIG. 10). The EIF2 signaling pathway associated with endoplasmic reticulum (ER) stress was significantly up-regulated in neutrophils from one-week and three-week LL2 mice compared to control neutrophils (FIG. 11A). Neutrophils from three-week LL2 mice had substantial up-regulation of multiple pathways associated with inflammatory responses, ROS and oxidative stress response, iNOS signaling, and ER stress compared to neutrophils from one-week LL2 mice (FIG. 4B). Most of these pathways have been previously implicated in PMN-MDSC function²⁸. Consistent with these results, genes regulated by XBP1, LPS, IL-1, HIF1A, TNF, and ATF4 were increased in three-week LL2 mice over one-week LL2 mice (FIG. 4C). These results suggested that BM neutrophils in three-week but not one-week LL2 mice could be bona fide PMN-MDSCs.

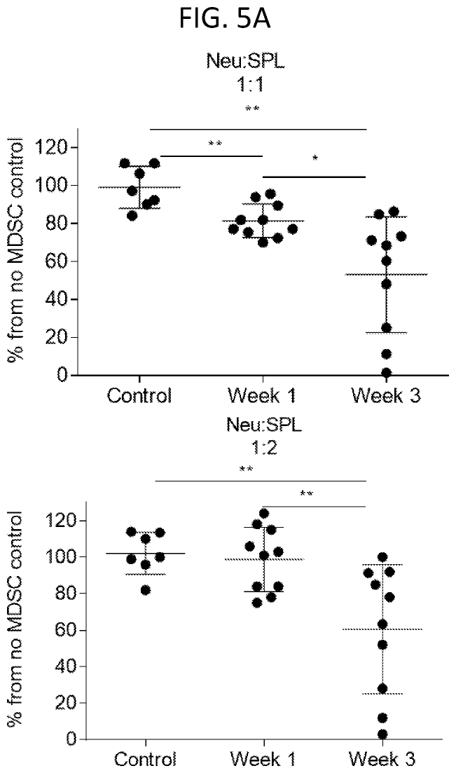

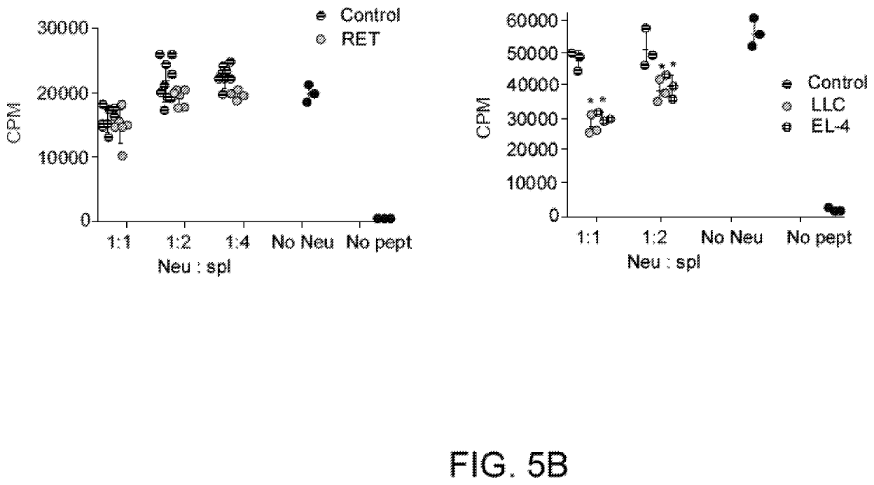

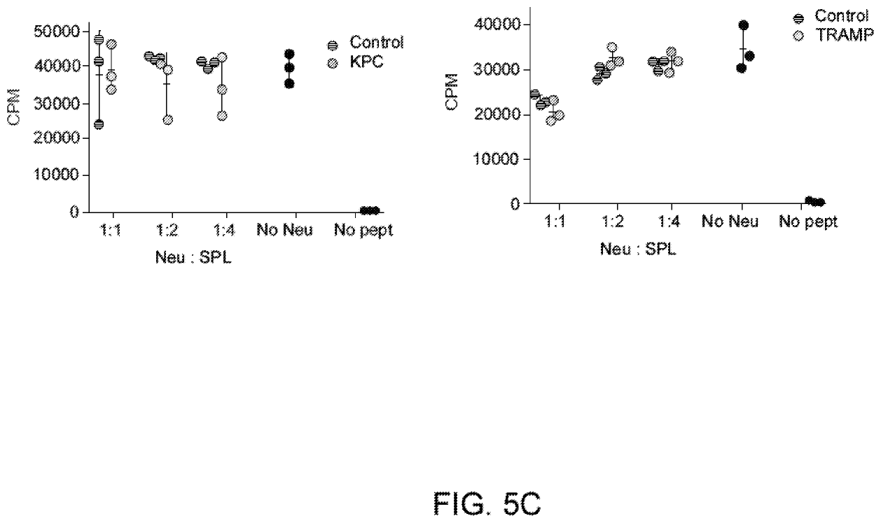

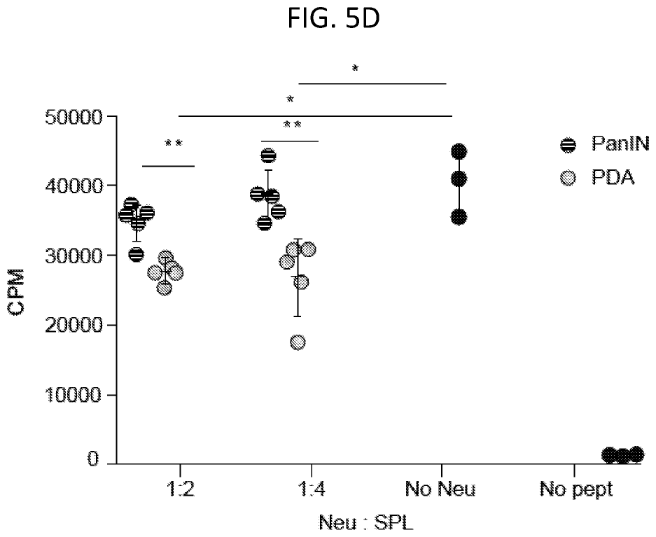

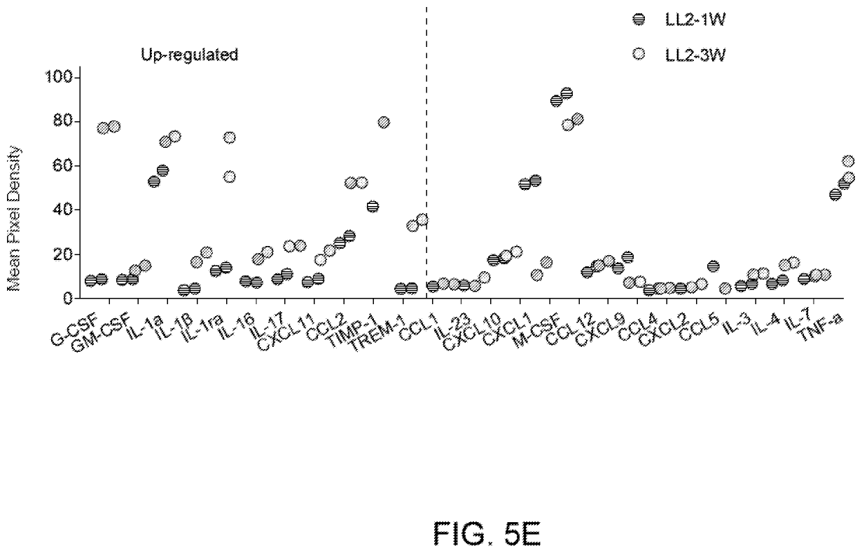

To functionally verify this observation, we evaluated the ability of BM neutrophils to suppress antigen-specific T cell responses. The neutrophils from one-week tumor-bearing mice showed minimal suppressive activity, whereas neutrophils from three-week tumor-bearing mice exhibited potent suppressive activity (FIG. 5A). BM neutrophils from control mice were not suppressive. Similar to the cells from one-week LL2 mice, BM neutrophils from RET melanoma mice (FIG. 5B), KPC and TRAMP mice (FIG. 5C) did not suppress T cell responses. In contrast, BM neutrophils from s.c. LLC and EL4 mice suppressed T cell proliferation (FIG. 5B). In a separate set of experiments, we compared suppression by BM neutrophils isolated from KPC mice with PanIN and PDA. Neutrophils from mice with PanIN had no suppressive activity. Neutrophils from mice with PDA had substantial suppressive activity (FIG. 5D).

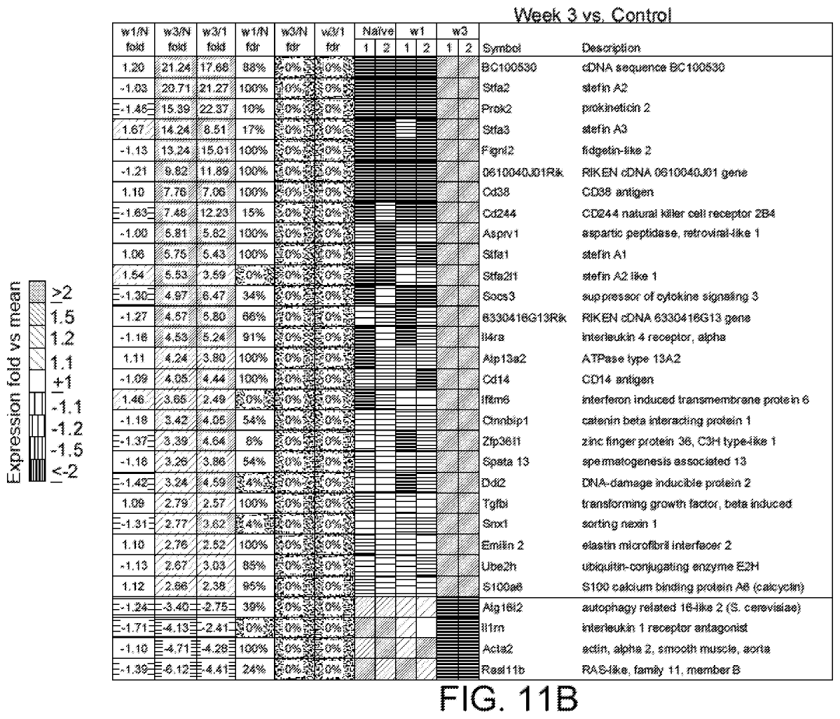

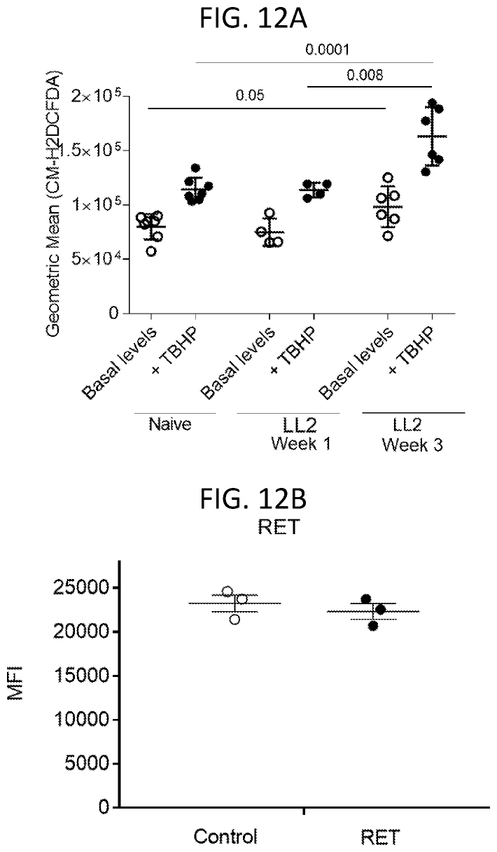

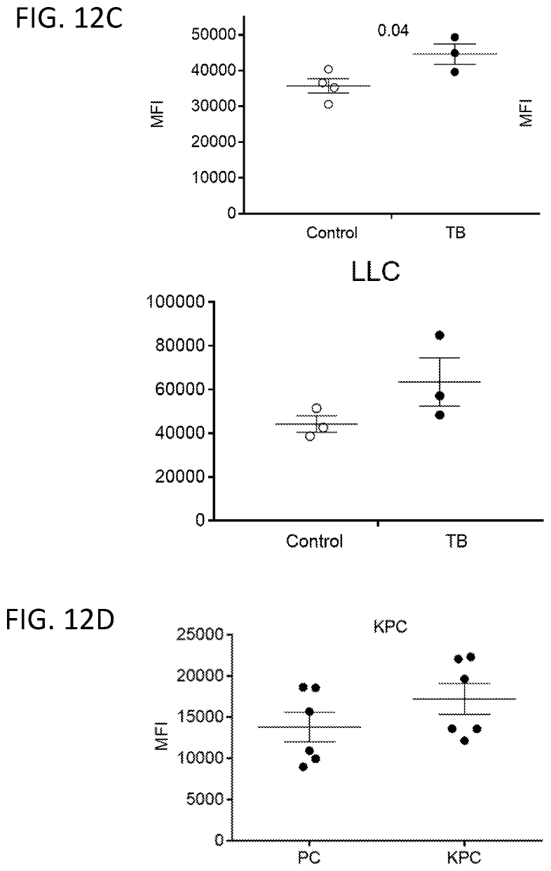

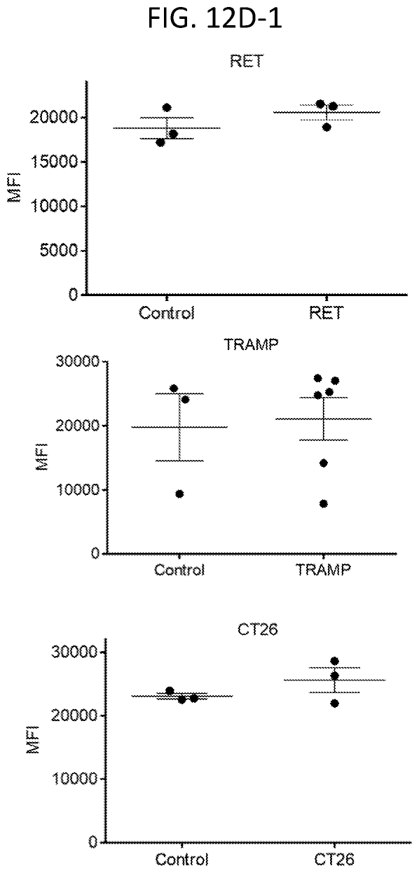

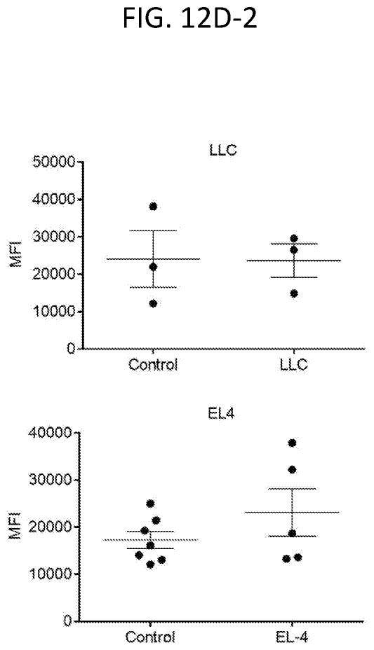

Since ROS production and the oxidized stress response were among the main pathways up-regulated in neutrophils in three-week LL2 TB mice, we evaluated the level of cytoplasmic ROS produced by neutrophils. BM neutrophils from three-week LL2 mice, but not from one-week LL2 mice, had markedly higher cytoplasmic ROS than control neutrophils (FIG. 12A). Neutrophils from RET melanoma mice had a ROS production similar to that of control mice (FIG. 12B), whereas BM neutrophils from s.c. EL4 and LLC mice had a slightly elevated ROS level (FIG. 12C).

To better understand the mechanism regulating functional differences in PMN at early and late stages of cancer we assessed the cytokine profile in sera of mice with one-week and three-week LL2 tumors. We observed up-regulation (more than 2 standard deviations) of number of pro-inflammatory cytokines (G-CSF, GM-CSF, IFN-γ, IL-1β, IL-17, TREM-1, TNF) in mice with three-week tumors as compared to one-week tumor (FIG. 5E).

Thus, based upon these functional characteristics, BM neutrophils from three-week LL2 orthotopic mice, and s.c. LLC and EL4 mice, and, to some extent, KPC mice with invasive PDA were typical PMN-MDSCs. BM neutrophils from one-week LL2 mice and from mice with GEMs of cancer were not PMN-MDSCs. However, they had elevated levels of ER stress response genes. We provisionally called them PMN-MDSC-Like Cells (PM-LCs). This term was coined previously to describe a population of activated neutrophils in cancer that did not acquire full capacity to suppress immune responses⁸.

### PM-LC and PMN-MDSC are Metabolically Distinct

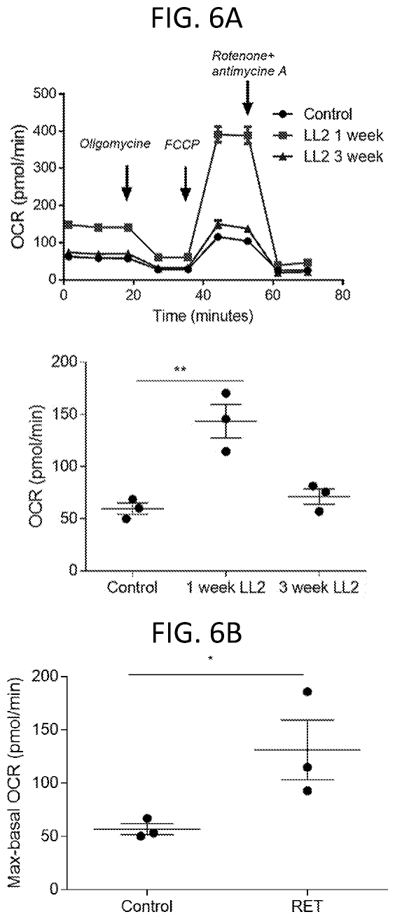

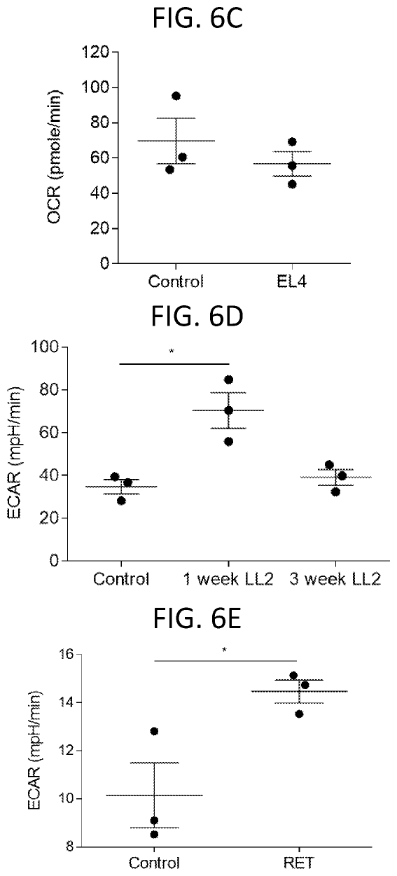

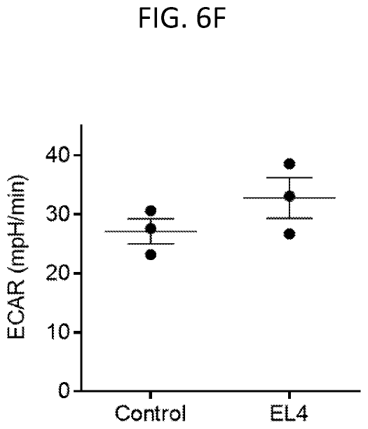

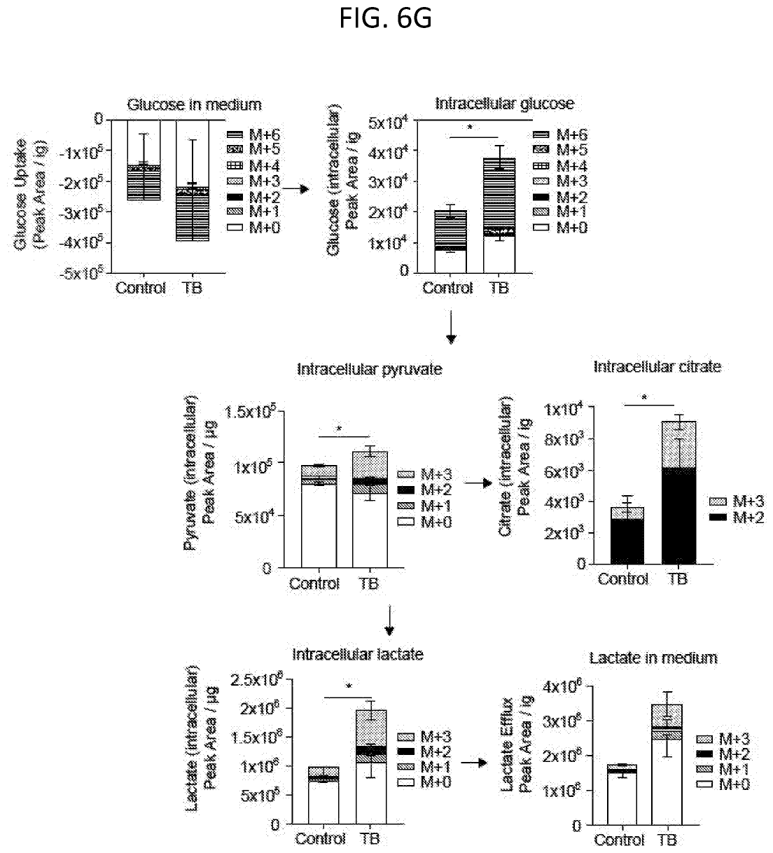

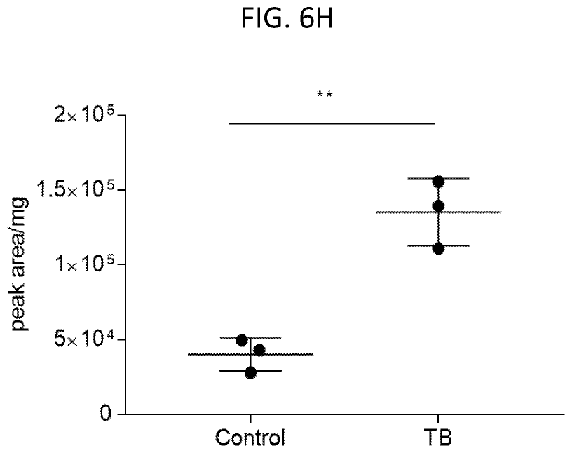

Next, we evaluated oxidative phosphorylation (OXPHOS) and glycolysis in BM neutrophils. We observed that BM PM-LCs from one-week LL2 mice had a markedly higher oxygen consumption rate (OCR) than control neutrophils, which is indicative of increased OXPHOS in these cells. In contrast, PMN-MDSCs from three-week LL2 mice had an OCR similar to control neutrophils (FIG. 6A). BM PM-LCs isolated from RET melanoma mice also demonstrated a significantly higher OCR compared to control neutrophils (FIG. 6B). In contrast, BM PMN-MDSCs from EL4 mice had OCR levels similar to that of control cells (FIG. 6C). PM-LCs from one-week LL2 mice also had a markedly higher extracellular acidification rate (ECAR) (FIG. 6D), which is indicative of increased glycolysis. BM PMN-MDSCs from three-week LL2 mice had ECAR similar to control cells. PM-LCs from RET melanoma mice also had a higher ECAR compared to control neutrophils (FIG. 6E). No differences were observed in BM PMN-MDSCs from EL4 mice (FIG. 6F). Thus, BM PM-LCs from the early stages of orthotopic lung cancer and from transgenic RET melanoma mice demonstrated increased OXPHOS and glycolysis compared to control neutrophils or PMN-MDSC.

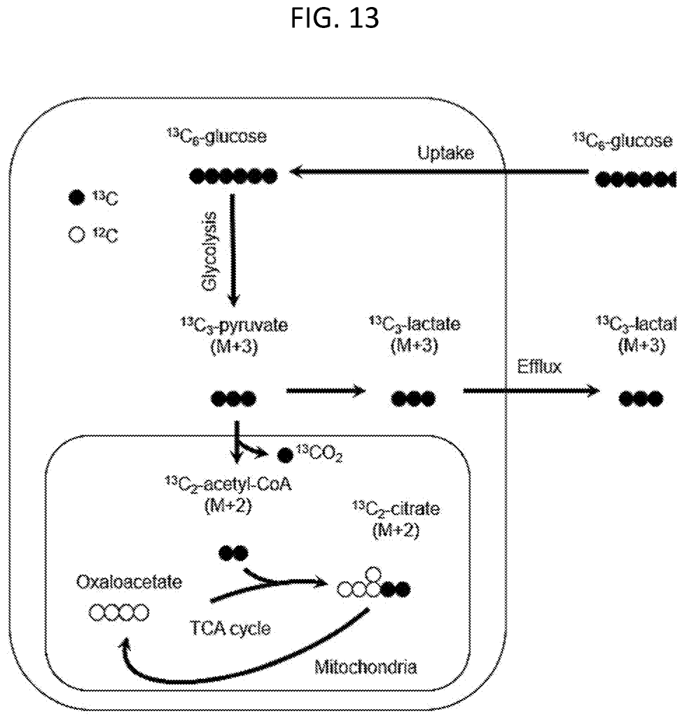

We then performed metabolomics analysis of ¹³C₆-glucose through glycolysis (FIG. 13) in control neutrophils and PM-LCs from one-week tumor-bearing mice. BM PM-LC had higher uptake of glucose along with higher intracellular levels of pyruvate, citrate, and lactate compared to control neutrophils. They also secreted markedly more lactate (FIG. 6G). In addition, PM-LC had much higher rates of incorporation of the ¹³C atoms into pyruvate (M+3), lactate (M+3), and citrate (M+2) (FIG. 6G). The ^{—}C-labeling patterns we observed in PM-LC strongly indicated that there was increased flux of glucose carbon through glycolysis and into the tricarboxylic acid (TCA) cycle in PM-LC. As a consequence of accelerated glycolysis and flux through the TCA cycle, PM-LC had significantly higher amounts of ATP (FIG. 6H). In contrast to PM-LC, metabolomics analysis of BM PMN-MDSCs from three-week s.c. LLC mice showed that PMN-MDSCs did not have higher ¹³C-labeling of pyruvate and lactate compared to neutrophils in control mice, nor were there any differences in the efflux of lactate into the media (FIG. 14A). There were also no differences observed in the level of ATP in neutrophils and PMN-MDSCs (FIG. 14B). These results were in agreement with the OCR and ECAR analyses where there were no differences in OXPHOS or glycolysis in PMN-MDSCs compared to control neutrophils. The increased metabolism of BM PM-LC was not associated with increased mitochondrial mass (FIG. 12D).

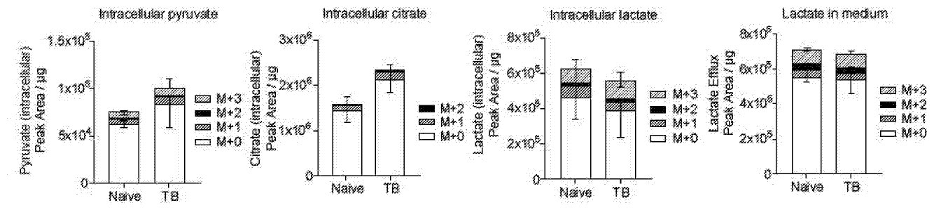

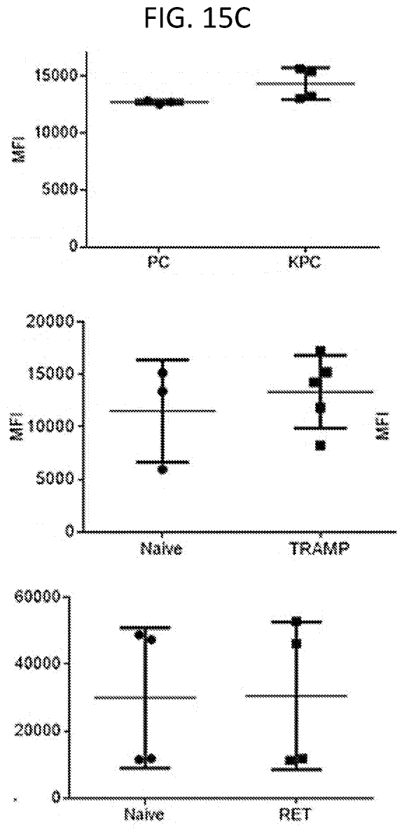

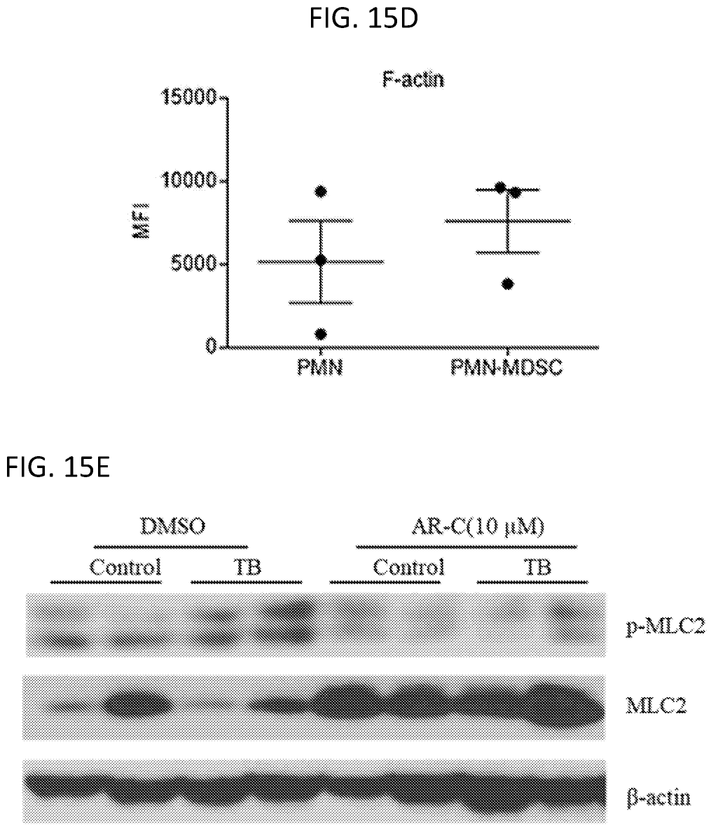

Because neutrophils have few mitochondria and primarily use glycolysis to generate ATP²⁹, we hypothesized that two major mechanisms could account for the increased glucose metabolic rate in PM-LCs: 1) an increase in the expression of glycolytic enzymes and 2) an increase in glucose uptake. PM-LC, but not PMN-MDSC, had increased expression of the Glut1 glucose transporter (FIG. 6C and FIG. 6D), which may explain the increased glucose uptake by PM-LCs. We did not observe differences in the expression of major glycolytic enzymes between control neutrophils and PM-LC from RET melanoma mice (FIG. 15A), which was consistent with the results obtained at RNA-Seq analysis. Thus, PM-LC had high glucose uptake, increased OXPHOS, increased TCA cycle flux, and increased glycolysis resulting in a substantially higher production of ATP compared to neutrophils from control mice, which may account for their increased migratory ability. This effect was absent in PMN-MDSCs.

### Increased Spontaneous Migration of PM-LC in Cancer is Regulated by ATP

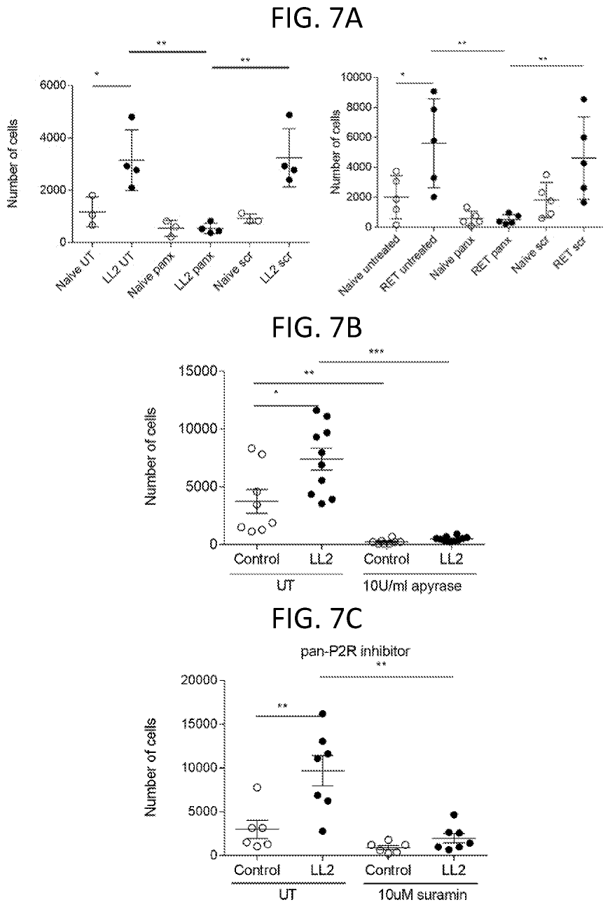

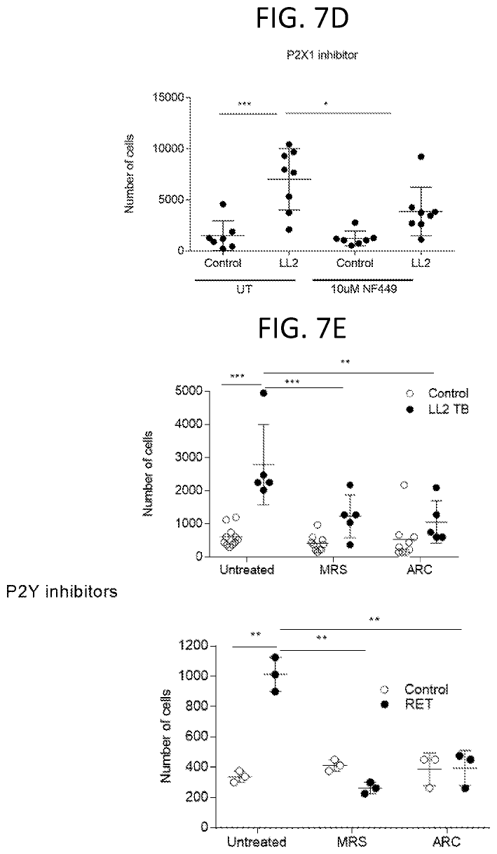

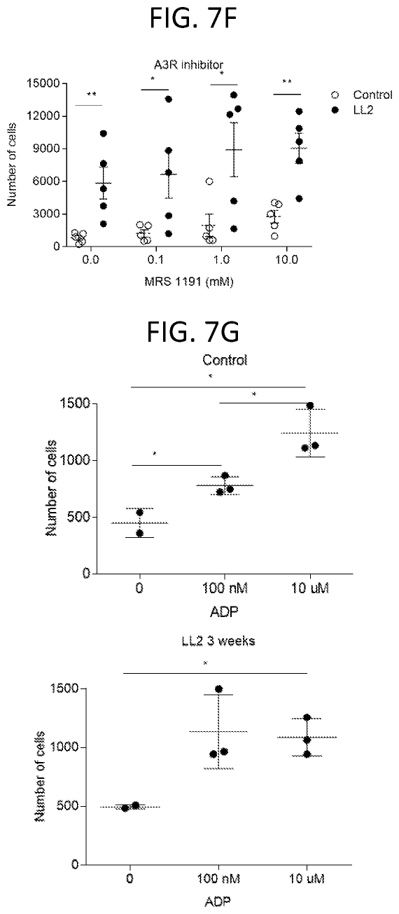

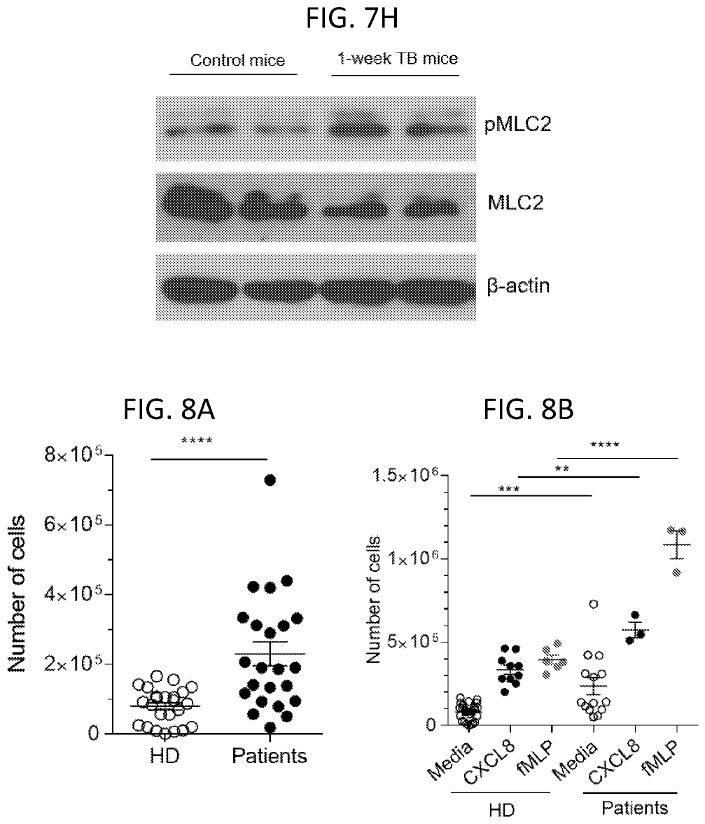

ATP-mediated autocrine signaling can enhance gradient sensing and chemotaxis of neutrophils³⁰. We hypothesized that BM PM-LC might use ATP-mediated autocrine signaling as a mechanism to support their enhanced spontaneous migration. ATP can be released from cells through mechanosensitive channels, such as pannexin-1 and connexin hemichannels^{31, 32}. We examined the effect of disrupting ATP release from the cell on the ability of PM-LCs to spontaneously migrate. We inhibited pannexin-1 hemichannels using ¹⁰Panx (panx), a pannexin-1 mimetic inhibitory small peptide. This resulted in the abrogation of increased spontaneous migration exhibited by PM-LC from RET melanoma and one-week LL2 TB mice (FIG. 7A). The specificity of the effect was confirmed with scrambled ¹⁰Panx, which had no effect on the spontaneous migration of PM-LCs (FIG. 7A). To verify that spontaneous migration is truly dependent upon ATP, we treated cells with apyrase, an enzyme that catalyzes the hydrolysis of ATP. Upon apyrase treatment, the spontaneous migration of both control neutrophils and PM-LCs was completely abrogated (FIG. 7B).

There are several families of ectonucleotidases expressed on the cell surface of mammalian cells that can hydrolyze ATP to ADP, AMP, and adenosine, and each of these metabolites can signal through a distinct set of purinergic receptors³³. Neutrophils have been reported to use the purinergic receptors P2X₁, P2Y₁, and P2Y₂, which bind ATP, and A3, which binds adenosine, to amplify chemotactic signals^{34, 35}. To determine if these receptors are involved in the spontaneous migration of PM-LCs, we treated cells with suramin, a pan-P2 receptor (pan-P2R) inhibitor and observed that spontaneous migration of PM-LCs was completely abrogated (FIG. 7C). To clarify the role of individual purinergic receptors, we treated cells with NF449, a P2X₁-specific inhibitor. The ability of PM-LCs to spontaneously migrate was only partially (50%) inhibited (FIG. 7D). However, blocking the P2Y₁ and P2Y₂ receptors using the selective inhibitors MRS2179 and AR-C118925XX, respectively, completely abrogated the increased spontaneous migration (FIG. 7E). In contrast, blocking the A3 receptor with the MRS 1191 inhibitor did not affect the migration of PM-LC (FIG. 7F). These data indicated that increased production of ATP by PM-LC in TB mice was directly involved in the increased spontaneous migration of these cells in a paracrine fashion. If increased production of ATP and paracrine signaling was a major mechanism of increased spontaneous migration of PM-LC, then activating the purinergic receptors should increase the spontaneous migration of control neutrophils and PMN-MDSC. To test this hypothesis we treated these cells with ADP and observed significantly increased migration (FIG. 7G).

Next, we investigated the signaling pathways by which ATP could affect spontaneous neutrophil migration. Neutrophil migration requires polarization of the front and rear of the cell, processes that are coordinated by the Rho guanosine triphosphatases (GTPases) Rac, Cdc42 and RhoA³⁶. Rac GTPase activation at the leading edge of the cell induces actin polymerization, and Cdc42 signaling can steer the direction of migration³⁷. We evaluated actin polarization (F-actin) in neutrophils. Both, CXCL1 and fMLP stimulation resulted in substantial increase in F-actin. However, no differences between unstimulated and stimulated BM neutrophils from control and PM-LC from RET, KPC, or TRAMP TB mice were found (FIG. 15B and FIG. 15C).

The rear of the cell generates the contractile force that is needed to overcome any cell adhesions and move the cell body forward. This process is regulated by activation of RHO-associated coiled-coil-containing protein kinase (ROCK), that phosphorylates Ser₁₉ on the myosin light chain 2 (MLC2)²¹. We evaluated the target ROCK activity pMLC2 and found that PM-LC from one-week LL2 TB mice had substantial up-regulation of pMLC2 as compared to control neutrophils (FIG. 7H). Selective P2Y₂inhibitor AR-C118925XX abrogated up-regulation of pMLC2 in PM-LC (FIG. 15E).

Increased Neutrophil Migration is Associated with Increased Tumor Spread

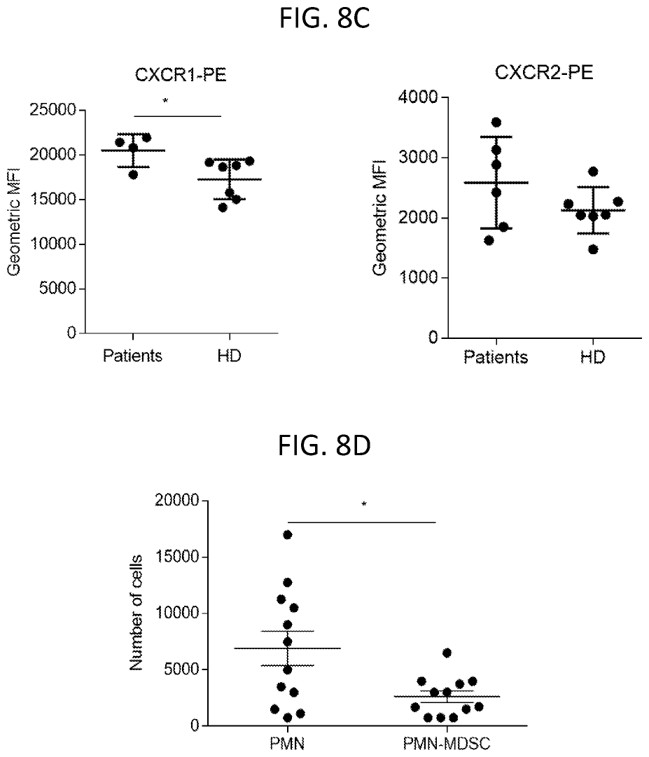

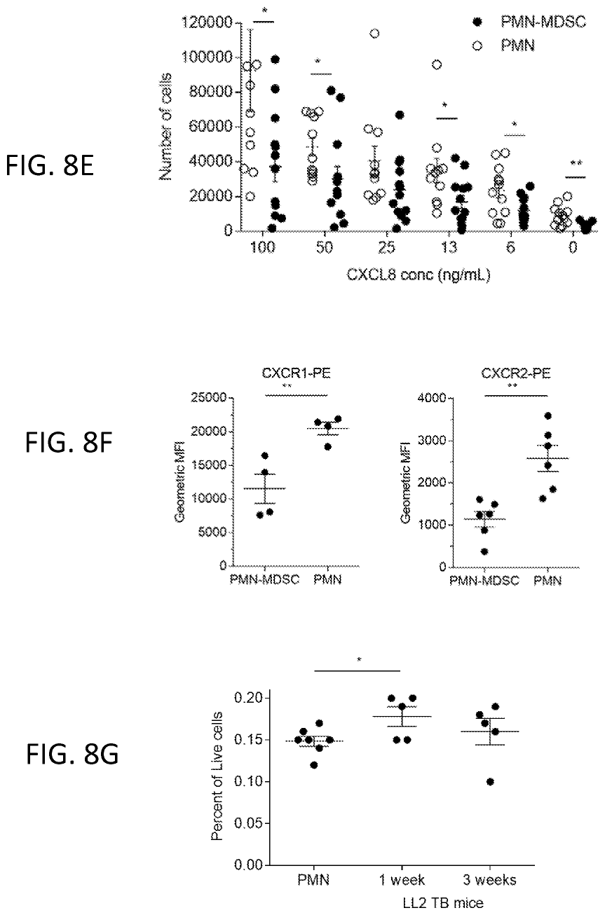

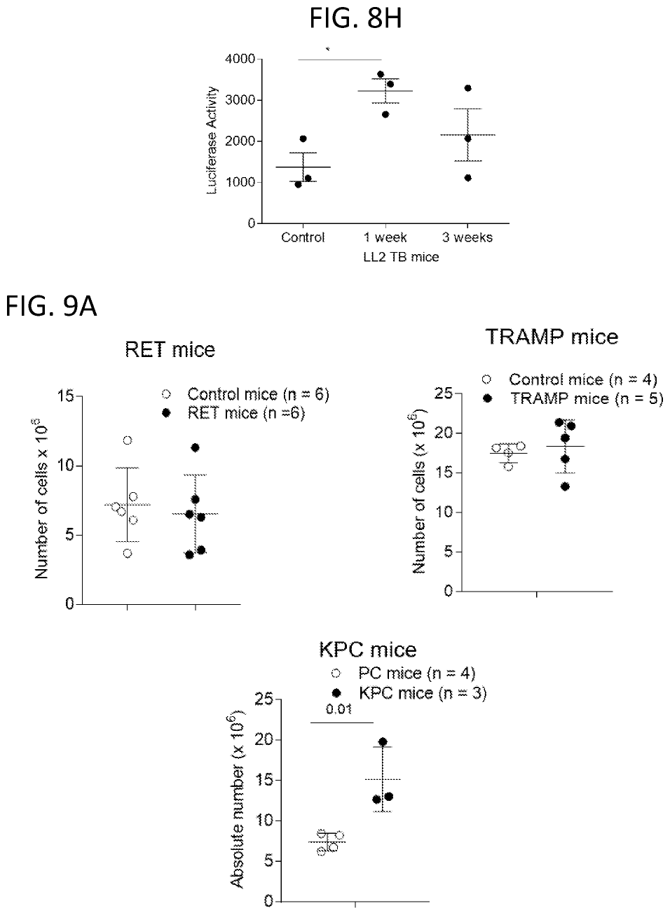

Next, we evaluated the migratory behavior of neutrophils from cancer patients. Neutrophils were isolated from peripheral blood by negative selection and their response to CXCL8 (IL-8) and fMLP stimulation was assessed. Neutrophils from cancer patients demonstrated markedly higher spontaneous migration compared to neutrophils obtained from healthy donors (FIG. 8A). This was associated with more potent response to both CXCL8 and fMLP (FIG. 8B). Neutrophils from healthy donors and cancer patients had similar expression of the chemokine receptors CXCR1 and CXCR2 (FIG. 8C). We compared the motility of neutrophils and PMN-MDSC from the same patients. CD15⁺PMN-MDSCs were isolated from the low density peripheral blood mononuclear cell (PBMC) using magnetic beads⁸. CD15³⁰ neutrophils were also isolated from the high-density fraction using magnetic beads³⁸. Neutrophils exhibited substantially higher spontaneous CXCL8 stimulated migration than PMN-MDSCs from the same patient (FIG. 8D and FIG. 8E). PMN-MDSCs were found to have lower expression levels of CXCR1 and CXCR2 (FIG. 8F), which may account for their lower response to CXCL8. Despite the marked difference in migration, neutrophils and PMN-MDSC had similar level of actin polymerization (FIG. 15D), a finding that is consistent with the results obtained in TB mice. These data suggest that neutrophils, but not PMN-MDSCs, in cancer patients have higher spontaneous and chemokine-induced migration than neutrophils from healthy donors.

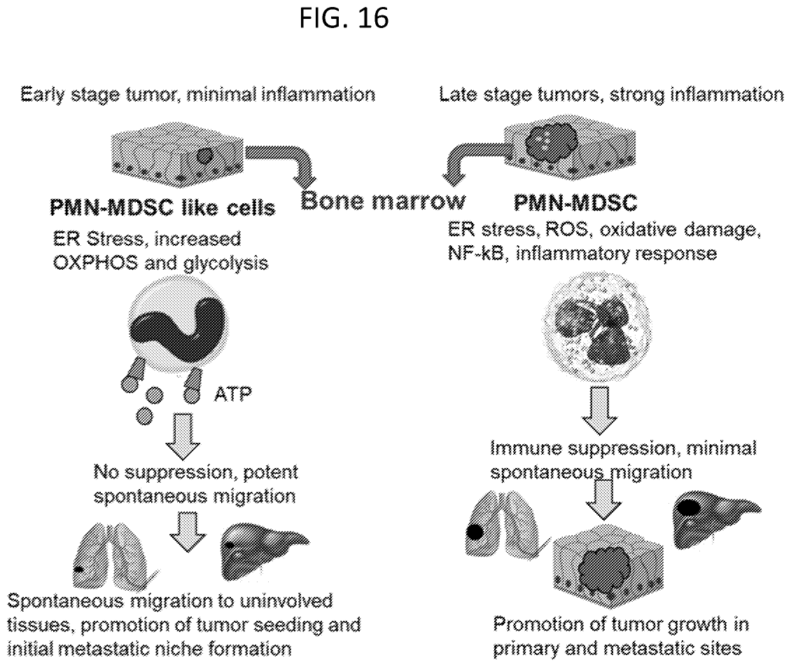

We asked whether the increased spontaneous motility of PM-LC could facilitate tumor-cell seeding. To test this hypothesis, we performed two types of experiments where we assessed the role of neutrophils in promoting tumor-cell seeding of lungs after i.v. injection. In the first group of experiments, BM neutrophils from control mice, PM-LC from one-week LL2 mice and PMN-MDSC from three-week LL2 mice were injected i.v. into tumor-free recipients 5 hours before the injection of CFSE-labeled LL2 tumor cells. The presence of tumor cells in lungs was evaluated 12 hours later. Administration of PM-LC but not PMN-MDSC caused significantly higher seeding of tumor cells compared to administration of control neutrophils (FIG. 8G). In the second group of experiments, control neutrophils, PM-LCs, and PMN-MDSCs were injected i.v. 5 hours prior to administration of LL2-Luc tumor cells and tumor growth was assessed 14 days later by measuring luciferase luminescence. All mice developed tumors. Administration of PMN-MDSC slightly increased tumor burden. However, it was significantly larger after administration of PM-LC (FIG. 8). Thus, PM-LC preferentially facilitate tumor-cell seeding, and it is possible that once PM-LC are in the tissue, these cells could facilitate tumor-cell metastasis (FIG. 16).

### EXAMPLE 3

### Discussion

Our study describes the association of the functional state of neutrophil activation in cancer with the ability of these cells to spontaneously migrate. It is likely that the population of neutrophils in tumor-bearing mice or cancer patients consists of classic neutrophils and a smaller population of pathologically activated immune-suppressive PMN-MDSCs. The abundance of PMN-MDSC depends on the stage and type of cancer^{s}. PMN-MDSC usually accumulate in advanced stage cancer and are often barely detectable in the early stages. Although PMN-MDSC may be detected in BM, their suppressive activity in BM is much lower than that of cells in the spleen and tumor site³. Our data confirmed the presence of PMN-MDSC in the BM of mice with different s.c. (ectopic) tumors and in mice with advanced orthotopic lung cancer. However, immune-suppressive BM PMN-MDSC were not found in mice with GEM of cancer and in early stage orthotopic lung cancer. It is possible that the degree of the inflammation associated with the different tumor models plays a role. Ectopic tumors that require injection of a relatively large number of tumor cells s.c. are associated with local inflammation that may facilitate rapid development of PMN-MDSCs. The same phenomenon is associated with late stage orthotopic or spontaneous cancers where the mice have a large tumor burden. BM neutrophils from mice with early stage orthotopic lung cancer and GEM of cancer did not have immune-suppressive activity. However, BM neutrophils in these models had elevated expression of genes in the ER stress pathway, one of the characteristics of pathological activation of these cells in cancer ³⁹. We found that these cells are markedly different from control BM neutrophils in their ability to spontaneously migrate. They also have increased expression of the genes associated with energy production, nucleic acid metabolism, increased OXPHOS and glycolytic rates, and considerably higher ATP compared to control neutrophils. Recently, to characterize pathologically activated myeloid cells that did not yet acquire potent suppressive activity, the term “MDSC-like” cells was introduced⁸. We used the term PMN-MDSC-like cells (PM-LC) to define the functional state of BM neutrophils from mice with early stage orthotopic lung cancer and GEM of cancer that did not have immune-suppressive activity.

Why do PM-LC have a potent ability to spontaneously migrate? In order to move, neutrophils must acquire spatial asymmetry. The crosstalk between the front and rear of the cell that maintain this polarization are poorly understood, but is believed to be maintained by coordinated Rho guanosine triphosphatase (GTPase) signaling between Rac, Cdc42 and RhoA³⁶. In migrating neutrophils, leading edge protrusion (lamellipod) formation is caused by the polymerization of monomeric globular actin (G-actin) into filamentous actin (F-actin). The rear of the cell (uropod) generates the contractile force that is needed to overcome any cell adhesions and move the cell body forward. Activation of RhoA leads to the activation of RHO-associated coiled-coil-containing protein kinase (ROCK), that directly phosphorylates Ser19 on the regulatory light chain of myosin, MLC2⁴⁰. We were focused on the two endpoints of the signaling cascades that result in neutrophil migration: F-actin polymerization and phosphorylation of MLC2. Although, as expected, F-actin was substantially increased in neutrophils in response to stimuli, no differences were observed between control neutrophils and PM-LC. In contrast, PM-LC had substantially higher level of pMLC2 implicating contractile force in the rear of cells as the driving force to support the increased spontaneous migration of PM-LC.

What drives increased migration of PM-LC? Our study demonstrated that BM PM-LC had increased ATP, whereas BM PMN-MDSCs had ATP levels similar to control neutrophils. This paralleled the changes in migratory capacity. The metabolism of MDSC in cancer is not well understood. In in vitro-generated MDSC, glycolysis increased concurrently with increased arginase I activity and activation of AMP-activated protein kinase, which can drive metabolism towards fatty acid oxidation (FAO)⁴¹. Tumor-infiltrating MDSC primarily used FAO and oxidative phosphorylation as their main metabolic pathway. In support of this finding, tumor-infiltrating MDSC increased fatty acid uptake and upregulated key FAO enzymes⁴². Our data demonstrate that increased metabolism and ATP in BM PM-LC precedes acquisition of the functional immunosuppressive characteristics of PMN-MDSC. Since BM PM-LC are the cells that preferentially migrate into tissues, our data are consistent with the observations on the metabolism of PMN-MDSC in tumors.

Recent studies have shown that neutrophils release ATP in response to chemokines and that autocrine signaling serves to amplify the chemotactic signal³⁰. ATP is released through connexin and pannexin-1 hemichannels³¹ and bind to three major families of purinergic receptors: P1, P2X, and P2Y⁴³. P2Y₂ and A3 are important mediators of extracellular ATP, and with CD39, aid in the polarization of neutrophils by translocating to the leading edge of the ce11³° ³¹. P2X1 was implicated in RhoA activation and phosphorylation of MLC2 in human and murine neutrophils⁴⁴. By blocking purinergic receptors and ATP hydrolysis in this study, we demonstrated a direct role for ATP to support increased spontaneous migration of PM-LC. At the same time, ADP, a ligand of purinergic receptors, substantially increased spontaneous migration of control neutrophils and PMN-MDSC, supporting the notion that ATP release may be sufficient to drive high PM-LC motility.

In line with our studies in mice, peripheral blood neutrophils from cancer patients exhibited dramatically higher spontaneous migration than neutrophils from healthy individuals. PMN-MDSC had substantially reduced migration, which was consistent with a previous report of a population of human PMN-MDSC with low migratory activity²³. Bona-fide PMN-MDSCs are capable of migrating towards a chemokine gradient, such as one leading to the tumor site, but have very poor ability to migrate to uninvolved tissues. There is now ample evidence that neutrophils can acquire features of PMN-MDSC in tissues of tumor-bearing hosts⁵. PM-LC may represent the first step of pathologic activation of neutrophils in cancer. These cells possess a potent ability to migrate to distant sites in the absence of inflammation or tumor cells compared to control neutrophils or PMN-MDSC.

### REFERENCES

1. Fridlender, Z. G. et al. Polarization of tumor-associated neutrophil phenotype by TGF-beta: “N1” versus “N2” TAN. Cancer Cell 16, 183-194 (2009).
2. Eruslanov, E. B. et al. Tumor-associated neutrophils stimulate T cell responses in early-stage human lung cancer. J Clin Invest 124, 5466-5480 (2014)
3. Veglia, F., Perego, M. & Gabrilovich, D. Myeloid-derived suppressor cells coming of age. Nat Immunol in press (2018).
4. Coffelt, S. B., Wellenstein, M. D. & de Visser, K. E. Neutrophils in cancer: neutral no more. Nat Rev Cancer 16, 431-446 (2016).
5. Zhou, J., Nefedova, Y., Lei, A. & Gabrilovich, D. Neutrophils and PMN-MDSC: Their biological role and interaction with stromal cells. Semin Immunol (2017).
6. Ortiz, M. L. et al. Immature myeloid cells directly contribute to skin tumor development by recruiting IL-17-producing CD4+ T cells. J Exp Med 212, 351-367 (2015).
7. Ortiz, M. L., Lu, L., Ramachandran, I. & Gabrilovich, D. I. Myeloid-derived suppressor cells in the development of lung cancer. Cancer Immunol Res 2, 50-58 (2014).
8. Bronte, V. et al. Recommendations for myeloid-derived suppressor cell nomenclature and characterization standards. Nat. Commun. 7: 12150 (2016).
9. Seyfried, T. N. & Huysentruyt, L. C. On the origin of cancer metastasis. Critical reviews in oncogenesis 18, 43-73 (2013).
10. Achberger, S. et al. Circulating immune cell and microRNA in patients with uveal melanoma developing metastatic disease. Mol Immunol 58, 182-186 (2013).
11. Weide, B. et al. Myeloid-derived suppressor cells predict survival of patients with advanced melanoma: comparison with regulatory T cells and NY-ESO-1- or melan-A-specific T cells. Clin Cancer Res 20, 1601-1609 (2014).
12. Condamine, T., Ramachandran, I., Youn, J. I. & Gabrilovich, D. I. Regulation of tumor metastasis by myeloid-derived suppressor cells. Annu Rev Med 66, 97-110 (2015).
13. Coffelt, S. B. et al. IL-17-producing gammadelta T cells and neutrophils conspire to promote breast cancer metastasis. Nature 522, 345-348 (2015).
14. Toh, B. et al. Mesenchymal transition and dissemination of cancer cells is driven by myeloid-derived suppressor cells infiltrating the primary tumor. PLoS biology 9, e1001162 (2011).
15. Liu, Y. et al. MicroRNA-494 Is Required for the Accumulation and Functions of Tumor-Expanded Myeloid-Derived Suppressor Cells via Targeting of PTEN. The Journal of Immunology 188, 5500-5510 (2012).
16. Yang, L. et al. Abrogation of TGF beta signaling in mammary carcinomas recruits Gr-1+CD11b+ myeloid cells that promote metastasis. Cancer Cell 13, 23-35 (2008).
17. Huh, S. J., Liang, S., Sharma, A., Dong, C. & Robertson, G. P. Transiently entrapped circulating tumor cells interact with neutrophils to facilitate lung metastasis development. Cancer Res 70, 6071-6082 (2010).
18. Ichikawa, M., Williams, R., Wang, L., Vogl, T. & Srikrishna, G. S100A8/A9 activate key genes and pathways in colon tumor progression. Mol Cancer Res 9, 133-148 (2011).
19. Kowanetz, M. et al. Granulocyte-colony stimulating factor promotes lung metastasis through mobilization of Ly6G+Ly6C+ granulocytes. Proc Natl Acad Sci USA 107, 21248-21255 (2010).
20. Kolaczkowska, E. & Kubes, P. Neutrophil recruitment and function in health and inflammation. Nat Rev Immunol 13, 159-175 (2013).
21. Mocsai, A., Walzog, B. & Lowell, C. A. Intracellular signalling during neutrophil recruitment. Cardiovasc Res 107, 373-385 (2015).
22. Kumar, V., Patel, S., Tcyganov, E. & Gabrilovich, D. I. The Nature of Myeloid-Derived Suppressor Cells in the Tumor Microenvironment. Trends Immunol (2016).
23. Brandau, S. et al. Myeloid-derived suppressor cells in the peripheral blood of cancer patients contain a subset of immature neutrophils with impaired migratory properties. J Leukoc Biol 89, 311-317 (2011).
24. Kato, M. et al. Transgenic mouse model for skin malignant melanoma. Oncogene 17, 1885-1888 (1998).
25. Hingorani, S. R. et al. Trp53R172H and KrasG12D cooperate to promote chromosomal instability and widely metastatic pancreatic ductal adenocarcinoma in mice. Cancer Cell 7, 469-483 (2005).
26. Greenberg, N. M. et al. Prostate cancer in a transgenic mouse. Proc Natl Acad Sci USA 92, 3439-3443 (1995).
27. Evans, R. A. et al. Lack of immunoediting in murine pancreatic cancer reversed with neoantigen. JCI insight 1 (2016).
28. Casbon, A. J. et al. Invasive breast cancer reprograms early myeloid differentiation in the bone marrow to generate immunosuppressive neutrophils. Proc Natl Acad Sci USA 112, E566-575 (2015).
29. Biswas, S. K. Metabolic Reprogramming of Immune Cells in Cancer Progression. Immunity 43, 435-449 (2015).
30. Chen, Y. et al. ATP release guides neutrophil chemotaxis via P2Y2 and A3 receptors. Science 314, 1792-1795 (2006).
31. Chen, Y. et al. Purinergic signaling: a fundamental mechanism in neutrophil activation. Sci Signal 3, ra45 (2010).
32. Junger, W. G. Purinergic regulation of neutrophil chemotaxis. Cell Mol Life Sci 65, 2528-2540 (2008).
33. Junger, W.G. Immune cell regulation by autocrine purinergic signalling. Nature reviews. Immunology 11, 201-212 (2011).
34. Chen, Y. et al. ATP Release Guides Neutrophil Chemotaxis via P2Y2 and A3 Receptors. Science 314, 1792-1795 (2006).
35. Lecut, C. et al. P2X₁ Ion Channels Promote Neutrophil Chemotaxis through Rho Kinase Activation. The Journal of Immunology 183, 2801-2809 (2009).
36. Hind, L. E., Vincent, W. J. & Huttenlocher, A. Leading from the Back: The Role of the Uropod in Neutrophil Polarization and Migration. Developmental cell 38, 161-169 (2016).
37. Yang, H. W., Collins, S. R. & Meyer, T. Locally excitable Cdc42 signals steer cells during chemotaxis. Nat Cell Biol 18, 191-201 (2016).
38. Condamine, T. et al. Lectin-type oxidized LDL receptor-1 distinguishes population of human polymorphonuclear myeloid-derived suppressor cells in cancer patients. Sci Immunol 1 (2016).
39. Condamine, T. et al. ER stress regulates myeloid-derived suppressor cell fate through TRAIL-R-mediated apoptosis. J Clin Invest 124, 2626-2639 (2014).
40. Tybulewicz, V. L. & Henderson, R. B. Rho family GTPases and their regulators in lymphocytes. Nat Rev Immunol 9, 630-644 (2009).
41. Hammami, I. et al. Immunosuppressive activity enhances central carbon metabolism and bioenergetics in myeloid-derived suppressor cells in vitro models. BMC Cell Biol 13, 18 (2012). 42. Hossain, F. et al. Inhibition of Fatty Acid Oxidation Modulates Immunosuppressive

Functions of Myeloid-Derived Suppressor Cells and Enhances Cancer Therapies. Cancer Immunol Res 3, 1236-1247 (2015).
43. Junger, W. G. Immune cell regulation by autocrine purinergic signalling. Nat Rev Immunol 11, 201-212 (2011).
44. Lecut, C. et al. P2X1 ion channels promote neutrophil chemotaxis through Rho kinase activation. J Immunol 183, 2801-2809 (2009).
45. Schug, Z. T. et al. Acetyl-CoA synthetase 2 promotes acetate utilization and maintains cancer cell growth under metabolic stress. Cancer Cell 27, 57-71 (2015).
46. Li, B. & Dewey, C. N. RSEM: accurate transcript quantification from RNA-Seq data with or without a reference genome. BMC bioinformatics 12, 323 (2011).
47. Love, M. I., Huber, W. & Anders, S. Moderated estimation of fold change and dispersion for RNA-seq data with DESeq2. Genome biology 15, 550 (2014).
48. Othmer, H. G., Dunbar, S. R. & Alt, W. Models of dispersal in biological systems. Journal of Mathematical Biology 26, 263 (1988).
49. Dunn, G. A. Characterising a kinesis response: time averaged measures of cell speed and directional persistence. Agents and Actions Supplements 12, 19 (1983).
50. Farrell, B. E., Daniele, R. P. & Lauffenburger, D. A. Quantitative relationships between single-cell and cell-population model parameters for chemosensory migration responses of alveolar macrophages to C5a. Cell motility and the cytoskeleton 16, 279-293 (1990).

### Sequence Listing Free Text

The following information is provided for sequences containing free text under numeric identifier <223>.

| SEQ ID NO: | |
|---|---|
| (containing free text) | Free text under <223> |
| 1 | <223> Glut1 Fwd primer |
| 2 | <223> Glut1Rev primer |
| 3 | <223> Glut3 Fwd primer |
| 4 | <223> Glut3 Rev primer |
| 5 | <223> HIF1alpha Fwd primer |
| 6 | <223> HIF1alpha Rev primer |
| 7 | <223> Hexokinase Fwd primer |
| 8 | <223> Hexokinase Rev primer |
| 9 | <223> Phosphofructokinase Fwd primer |
| 10 | <223> Phosphofructokinase Rev primer |
| 11 | <223> GAPDH Fwd prime |
| 12 | <223> GAPDH Rev primer |
| 13 | <223> Phosphoglycerate kinase Fwd primer |
| 14 | <223> Phosphoglycerate kinase Rev primer |
| 15 | <223> Enolase Fwd primer |
| 16 | <223> Enolase Rev primer |
| 17 | <223> Pyruvate kinase Fwd primer |
| 18 | <223> Pyruvate kinase Rev primer |
| 19 | <223> Beta-actin Fwd primer |
| 20 | <223> Beta-actin Rev primer |

All publications cited in this specification and priority document U.S. Provisional Patent Application No. 62/745,903, filed Oct. 15, 2018, are incorporated herein by reference. While the invention has been described with reference to particular embodiments, it will be appreciated that modifications can be made without departing from the spirit of the invention. Such modifications are intended to fall within the scope of the appended claims.

1. (canceled)

2. A method for treating cancer comprising administering an effective amount of an agent that alters neutrophil migration to reduce or inhibit tumor cell seeding, tumor metastasis, or metastatic niche formation in the subject.

3. The method of claim 2, wherein the agent is an siRNA, shRNA, antibody or functional antigen-binding fragment.

4. The method of claim 2, wherein the subject has been diagnosed with leukemia, lymphoma, myeloma, bone marrow cancer, an epithelial cancer, breast cancer, lung cancer, prostate cancer, colorectal cancer, brain cancer, endometrial cancer, esophageal cancer, stomach cancer, bladder cancer, kidney cancer, pancreatic cancer, cervical cancer, head and neck cancer, ovarian cancer, melanoma, leukemia, myeloma, lymphoma, glioma, Non-Hodgkin's lymphoma, leukemia, multiple myeloma, or multidrug resistant cancer.

5. The method of claim 2, wherein the agent specifically binds or inhibits a chemokine, chemokine receptor, or chemoattractant.

6. The method of claim 5, wherein the chemokine receptor is CXCR1 of CXCR2.

7. The method of claim 5, wherein the agent specifically binds or inhibits fMLP, CXCL8, or CXCL1.

8. The method of claim 2, wherein the agent is (a) an inhibitor or antagonist of pannexin-1 or connexin hemichannels and/or (b) a peptide mimetic, nucleic acid molecule, small molecule compound, antibody, or a derivative thereof.

9. (canceled)

10. The method of any claim 2, wherein the agent alters glycolysis and/or block or inhibits Glut1 function.

11. (canceled)

12. The method of claim 2, wherein the agent blocks or inhibits a purinergic receptor, wherein the purinergic receptor is P2X₁, P2Y₁, or P2Y₂.

13. (canceled)

14. The method of claim 12, wherein the agent is one or more of suramin, NF449, MRS2179, AR-C118925, BMS-884775, and GLS-409, or a salt or derivative thereof.

15. The method of claim 2, wherein the agent reduces neutrophil ATP production or signaling.

16. (canceled)

17. The method of claim 2, wherein the agent reduces or inhibits the ER stress response.

18. The method of claim 17, wherein the agent is a small molecule modulator of B-I09.

19. The method of claim 17, comprising administering one or more of CDN-1163, a small molecule modulator of SERCA2b, TDI-194, AC-915, RX-1, ADoPep-2, and AC-2010, or a salt or derivative thereof.

20. The method of claim 2, comprising administering an antagonist or inhibitor of the expression, activity or activation of one or more of sXBP1, DDIT3 (CHOP), ATF4, ATF3, SEC61A, ARGI or NOS-2.

21-23. (canceled)

24. A method of determining a treatment regimen for a subject having cancer, said method comprising obtaining a sample from the subject and assessing the sample for the presence of PM-LC by a method comprising:
a) detecting one or more of genes or pathways identified in Table 1, FIG. 4B, FIG. 4C, FIG. 10, FIG. 11A, and FIG. 11B; or
b) detecting cell surface expression of one or more of Glut1, CXCR1, CXCR2, CD15, CD14, CD11b, CD33 and CD66b,
wherein said PM-LC are distinguishable from PMN-MDSC and wherein the presence of PM-LC is indicative of an increased likelihood of metastasis, whereupon treatment is altered.

25. A method of providing a personalized treatment regimen for a subject with cancer, said method comprising the steps of:
determining whether the cancer has metastasized; and, if no metastasis is detected,
determining whether PM-LC cells are present in a sample obtained from the patient by:
a) detecting one or more of genes or pathways identified in Table 1, FIG. 4B, FIG. 4C, FIG. 10, FIG. 11A, and FIG. 11B; or
b) detecting cell surface expression of one or more of Glut1, CXCR1, CXCR2, CD15, CD14, CD11b, CD33 and CD66b, and
if the presence of PM-LC cells is detected, then administering a metastatic treatment regimen.

26. The method of claim 24, further comprising detecting one or more of levels of expression of Glut1, oxidative phosphorylation, glucose uptake, TCA cycle flux, glycolysis, ATP production, motility or migration, spontaneous migration, cytoplasmic ROS, the ER stress response, antigen-specific T cell suppression, myosin light chain 2 phosphorylation, and chemotaxis in response to CXCL8.

27. The method of claim 24, further comprising detection of P2Y1 and/or P2Y2 receptors to identify PM-LC in said sample.

28. (canceled)

29. The method of claim 24, wherein
(a) the biological sample is bone marrow, peripheral blood, or tissue obtained from a lymph node, lung, or spleen;
(b) the subject has been diagnosed with leukemia, lymphoma, myeloma, bone marrow cancer, an epithelial cancer, breast cancer, lung cancer, prostate cancer, colorectal cancer, brain cancer, endometrial cancer, esophageal cancer, stomach cancer, bladder cancer, kidney cancer, pancreatic cancer, cervical cancer, head and neck cancer, ovarian cancer, melanoma, leukemia, myeloma, lymphoma, glioma, Non-Hodgkin's lymphoma, leukemia, multiple myeloma, or multidrug resistant cancer; and/or
(c) said treatment regimen includes administration of an effective amount of an agent that alters neutrophil migration to reduce or inhibit tumor cell seeding, tumor metastasis, or metastatic niche formation in a subject.

30-32. (canceled)
